(19) United States
(12) Patent Application Publication
(10) Pub. No.: US 2003/0124102 A1
(43) Pub. Date: Jul. 3, 2003

(54) Pax-encoding vector and use thereof

(76) Inventors: Michael A. Rudnicki, Gloucester (CA); Patrick Seale, Ottawa (CA)

Correspondence Address: CLARK & ELBING LLP, 101 FEDERAL STREET, BOSTON, MA, 02110, US

(21) Appl. No.: 10/245,171

(22) Filed: Sep. 17, 2002

Related U.S. Application Data
(60) Provisional application No. 60/322,923, filed on Sep. 17, 2001.

(51) Int. Cl. A61K 48/00; C12N 5/08; C12N 15/85

(52) U.S. Cl. 424/93.21; 435/455; 435/366

(57) ABSTRACT

The present invention provides a Pax-encoding vector that comprises a sequence encoding a Pax7, Pax3 or an active variant or fragment thereof, which can be used to induce myogenic differentiation of adult pluripotent stem cells. The present invention further pertains to methods of preparing the Pax-encoding vector. Also provided is a method of inducing myogenic differentiation of adult pluripotent stem cells.

### CROSS-REFERENCES TO RELATED APPLICATIONS

This application claims the benefit of U.S. provisional application U.S. Ser. No. 60/322,923, filed Sep. 17, 2001.

### FIELD OF THE INVENTION

The present invention pertains to the field of Pax-encoding vectors and more particularly to vectors comprising sequences that encode Pax7, Pax3, and/or biologically active variants or fragments thereof, and their use to induce differentiation of adult pluripotent stem cells to produce myoblasts.

### BACKGROUND

Myoblasts are precursor cells of the mesoderm that are destined for myogenesis. The determined myoblasts are capable of recognising and spontaneously fusing with other myoblasts leading to the production of a differentiated myotube. The multinucleated myotube no longer divides or synthesises DNA but produces muscle proteins in large quantity. These include constituents of the contractile apparatus and specialised cell-surface components essential to neuromuscular transmission.

Eventually, the differentiated muscle cell exhibits characteristic striations and rhythmic contractions. A further step in this pathway is maturation; the contractile apparatus and muscle at different stages of development contain distinct isoforms of muscle proteins such as myosin and actin, encoded by different members of multigene families.

Myoblasts have the potential for being used in a variety of ways. For example, the myoblasts may serve as vehicles for cell therapy, where one or more genes may be introduced into the myoblasts to provide a product of interest. In order to find wide utility in therapeutic applications, however, it will be necessary to develop methods for the sustained production by myoblasts of the product of interest.

Myoblasts are thought to be capable of repairing damaged or injured myofibers (Mauro, A., J. Biophys. Biochem. Cytol., 9: 493-495 (1961); Bischoff, R., in Mvology, Engel, A. G. and Franzini-Armstrong, C., Eds., New York: McGraw Hill, pp. 97-119,1994; and Grounds, M., Adv. Exp. Med. Biol., 280: 101-104 (1990)). Because myoblasts are thought to be capable of repairing damaged or injured myofibers, the technique of myoblast transfer (myoblast transplantation) has been proposed as a potential therapy or cure for muscular diseases, including Duchenne muscular dystropy (DMD).

Myoblast transfer involves injecting myoblast cells into the muscle of a mammal, particularly a human patient, requiring treatment. Although developed muscle fibres are not regenerative, the myoblasts are capable of a limited amount of proliferation, thus increasing the number of muscle cells at the location of myoblast infusion. Myoblasts so transferred into mature muscle tissue will proliferate and differentiate into mature muscle fibres. This process involves the fusion of mononucleated myoeenic cells (myoblasts) to form a multinucleated syncytium (myofiber or myotube). Thus, it has been proposed that muscle tissue which has been compromised either by disease or trauma may be supplemented by the transfer of myoblasts into the compromised tissue.

Moreover, cell cultures are widely used as in vitro models for studying the events involved during in vivo cellular or tissular development. For example, muscular development events can be reproduced during the myogenic differentiation of stem cell cultures. Accordingly, permanent mammalian cell cultures, especially human myogenic cell cultures, would be of considerable value for providing useful tools for dissecting the molecular and biochemical cellular events, for identifying and testing new drugs for muscular diseases, such as dystrophies, for the study of myogenesis, etc.

The “paired-box” family of transcription factors is intimately involved in the control of embryonic development. Different members of the Pax-family of transcription factors appear to regulate the development and differentiation of diverse cell lineages during embryogenesis (see Table 1) (Mansouri et al., 1999; Mansouri et al., 1994; Noll, 1993; Strachan and Read, 1994). Pax7 and the closely related Pax3 gene belong to a paralogous subgroup of Pax genes based on similar protein structures and partially overlapping expression patterns during mouse embryogenesis (Goulding et al., 1991; Jostes et al., 1990). Interestingly, the closely related Pax3 gene plays an essential role in regulating the developmental program of MyoD-dependent migratory myoblasts during embryogenesis (Maroto et al., 1997; Tajbakhsh et al., 1997).

Pax7 and Pax3 proteins bind identical sequence-specific DNA elements suggesting that they regulate similar sets of target genes (Schafer et al., 1994). Furthermore, increased expression and gain-of-function mutations in both Pax3 and Pax7 are associated with the development of alveolar rhabdomyosarcomas indicating that both molecules regulate similar activities in myogenic cells (Bennicelli et al., 1999). However, Pax7 but not Pax3 is expressed in adult human primary myoblasts (Schafer et al., 1994). Interestingly, differential expression of alternatively spliced Pax7 transcripts correlates with muscle regenerative efficiency in different strains of mice (Kay et al., 1998; Kay et al., 1997; Kay et al., 1995; Kay and Ziman, 1999).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide Pax7-encoding vectors and use thereof. In accordance with an aspect of the present invention, there is provided a vector comprising an expression cassette comprising a sequence encoding a Pax protein, wherein the Pax protein is selected from the groups consisting of: Pax7; Pax3; an active variant of Pax 7; an active variant of Pax 3; an active fragment of Pax 7; and an active fragment of Pax 7, and wherein the Pax protein can induce myogenic differentiation of adult pluripotent stem cells..

In accordance with another aspect of the invention, there is provided a method of differentiating adult pluripotent stem cells to produce myoblasts comprising the step of transforming or infecting the stem cells with a vector comprising an expression cassette comprising a sequence encoding a Pax protein, wherein the Pax protein is selected from the groups consisting of: Pax7; Pax3; an active variant of Pax 7; an active variant of Pax 3; an active fragment of Pax 7; and an active fragment of Pax 7.

In accordance with another aspect of the invention, there is provided use of myoblasts produced according to the methods described herein for transplantation in a mammal in need of such therapy.

### BRIEF DESCRIPTION OF THE FIGURES

The file of this patent contains at least one drawing executed in color. Copies of this patent with color drawings will be provided by the Patent and Trademark Office upon request and payment of the necessary fees.

FIG. 1 demonstrates that Pax7 is expressed specifically in proliferating myoblasts. (A) Pax7 was expressed at high levels in proliferating wild-type myoblasts (Wt-Mb) and MyoD-deficient cells (MyoD^{-/-} Mb) cells and down regulated in response to differentiation conditions (Wt-D and MyoD^{-/-} D). (B) Expression of Pax7 was specific to myogenic cells with low levels detected in C2C12 myoblasts. (C) Pax7 was not detected in RNA from a panel of tissues.

FIG. 2 depicts the expression of Pax7 in muscle satellite cells. (A) In situ hybridisation revealed that Pax7 mRNA was expressed at a frequency and location consistent with specific expression in satellite cells and myogenic precursor cells. (B) Pax7 expression was associated with PI positive nuclei (40× magnification). (C,D) High magnification (200×) of Pax7 expressing cell in wild type muscle was characteristic of a satellite cell residing beneath the basal lamina. (E,F) Increased numbers of cells expressed Pax7 in regenerating mdx muscle (40×). Black and white arrowheads indicate cells stained positive for Pax7 mRNA, and PI positive nuclei respectively. (PI: propidium iodide).

FIG. 3 demonstrates that Pax₇^{-/-} mice exhibit skeletal muscle deficiency. (A) Seven-day-old Pax7 mutant animals were approximately one-half the weight of wild type animals and had splayed hind limbs and an abnormal gait. (B,C) Hematoxylin-Eosin (HE) stained tibialis anterior muscle sections (40×) revealed a normal histological appearance of (C) Pax7 mutant muscle but fibre diameter was reduced 1.5 fold as compared to (B) wild type muscle. (D,E) The diaphragm of (E) mutant animals shown here in cross-section was significantly thinner than in (D) wild type animals (40×).

FIG. 4 depicts the absence of myoblasts in cultures derived from Pax7^{-/-} muscle. (A-J) Primary cell cultures were analysed by (A,F) phase microscopy; and immunocytochemistry with (B,G) anti-desmin and (D,I) anti-c-Met antibodies. (C,E,H,J) Cells stained with antibodies were counter-stained with Hoechst 33342 to show all nuclei. Black arrowheads depict satellite cell derived myoblasts in (A). White arrowheads indicate immunoreactive cells and corresponding nuclei in (B-E).

FIG. 5 depicts the complete ablation of satellite cells in Pax7^{-/-} muscle. (A-D) Transmission electron micrographs of 7-10 day old Pax7^{+/+} and (E,F) Pax7^{-/-} muscle. (A,C) Satellite cells (SC) are readily identified in Pax7^{+/+} muscle (7500×). (B,D) High magnification of satellite cells clearly revealed the plasma membrane (black arrowheads) separating the satellite cell from its adjacent myofiber, the continuous basal lamina surrounding the satellite cell and myofiber and the heterochromatic appearance of the nucleus (20,000×). (E,F) Myonuclei (fiber nuclei) (MN) but not satellite cells were present in Pax7 mutant muscles. Other ultrastructural differences were not detected.

FIG. 6 demonstrates the enhanced hematopoietic potential of Pax7^{-/-} muscle-derived pluripotent stem cells. (A-D) FACS analysis of hoechst stained muscle-derived cells demonstrated approximately equal numbers of verapamil sensitive side-population (SP) cells in both (A,B) Pax7^{+/+} and (C,D) Pax7^{-/-} muscles. (E) Myosin heavy chain positive muscle colonies predominate in stem cell medium/methylcellulose cultures of Pax7^{+/+} muscle cells. (F) Pax7^{-/-} muscle cells have increased hematopoietic potential and generate granulocyte and monocyte colonies verified by (G,H) Ly-6G immunoreactivity. (I) Colony forming assay of muscle cells cultured in stem cell medium/methylcellulose over a period of two weeks demonstrated almost a 10-fold increased hematopoietic potential of Pax7 mutant stem cells. Other cells represent both fibroblasts and adipocytes.

FIG. 7 is a schematic representation of the role of Pax7 in the specification of satellite cells. Muscle-derived pluripotent stem cells primarily give rise to myoblasts when cultured in stem cell medium. By contrast, Pax7^{-/-} muscle stem cells exhibit almost a 10-fold increase in propensity towards hematopoietic differentiation and are incapable of forming adult myoblasts. These data therefore implicate Pax7 in regulating the specification of adult muscle satellite cells by restricting the fate of pluripotent stem cells. Taken together, these experiments suggest the following hypothesis. Pluripotent stem cells (msc) within muscle represent the progenitors of sublaminar satellite cells that are specified following induction of Pax7. Satellite cells are subsequently activated in response to physiological stimuli to generate daughter myogenic precursor cells (mpc) prior to terminal differentiation into new or previously existing fibres.

FIG. 8 provides a demonstration of myogenic specification of SP cells. Fractionated SP cells infected with Ad-empty control virus (mock) and Ad-Pax7 virus (Ad-Pax7d) were analysed for expression of desmin and counter-stained with DAPI to show all nuclei.

FIG. 9 depicts the structure of an exemplary adenovirus-Pax7. Pax7 is expressed under the control of the murine CMV promoter (mCMV). The SV40 poly A (SVpA) sequence is downstream of the cDNA.

FIG. 10 depicts western analysis of Ad-Pax7 infected Cells. C2C12 myoblasts or 10T1/2 fibroblasts were infected with either Ad-Pax7 or Ad-empty. Western analysis indicates that Pax7 protein is expressed at high levels from the recombinant Ad-Pax7 virus. C2C12 myoblasts expressed low-levels of endogenous Pax7.

FIG. 11 provides a demonstration of myogenic specification of SP cells. Fractionated SP cells infected with Ad-empty control virus (A,B) and Ad-Pax7 virus (C-H) were analysed for expression of desmin (A,C,E,G) and counter-stained with DAPI to show all nuclei (B,D,F,H).

FIG. 12 depicts induction of Myf5lacZ by Pax7. Muscle-derived cells from Myf5nlacZ transgenic mice were infected with Ad-empty (A,B) or Ad-Pax7 (C-F). Expression of Pax7 resulted in up-regulation of Myf5nLacz indicating entry into the myogenic differentiation program.

FIG. 13, depicts the amino acid sequence of a human Pax7 protein (NCBI Accession number NM_{—}002584).

FIG. 14 depicts the amino acid sequence of variants of the human Pax7 protein (A NCBI Accession number NP_{—}002575; B NCBI Accession number NM_{—}013945).

FIG. 15 depicts the amino acid sequence of a long splice form of human Pax7 protein (NCBI Accession number S78502).

FIG. 16 depicts the amino acid sequence of a human Pax7 protein (NCBI Accession number CAA16432).

FIG. 17 depicts the amino acid sequence of a fragment of a human Pax7 protein (NCBI Accession number S50115).

FIG. 18 depicts the amino acid sequence of a chicken Pax7 protein (NCBI Accession number BAA23005).

FIG. 19 depicts the amino acid sequence of a human Pax3 protein (NCBI Accession number P23760)

FIG. 20 depicts the amino acid sequence of a human Pax3A protein (NCBI Accession number NP_{—}000429).

FIG. 21 depicts the amino acid sequence of a human Pax3B protein (NCBI Accession number NP_{—}039230).

FIG. 22 depicts the amino acid sequence of a human Pax3 protein (NCBI Accession number AAA03628).

FIG. 23 depicts the amino acid sequence of a mouse Pax3 protein (NCBI Accession number NP_{—}032807).

FIG. 24 depicts the amino acid sequence of a chicken Pax3 protein (NCBI Accession number AH004319)

### DETAILED DESCRIPTION OF THE INVENTION

Unless defined otherwise, all technical and scientific terms used herein have the same meaning as commonly understood by one of ordinary skill in the art to which this invention belongs.

Characterisation and Preparation of Pax-Encoding Vectors

One embodiment of the present invention provides a vector comprising an expressible sequence encoding Pax7, Pax3 or an active variant or fragment thereof.

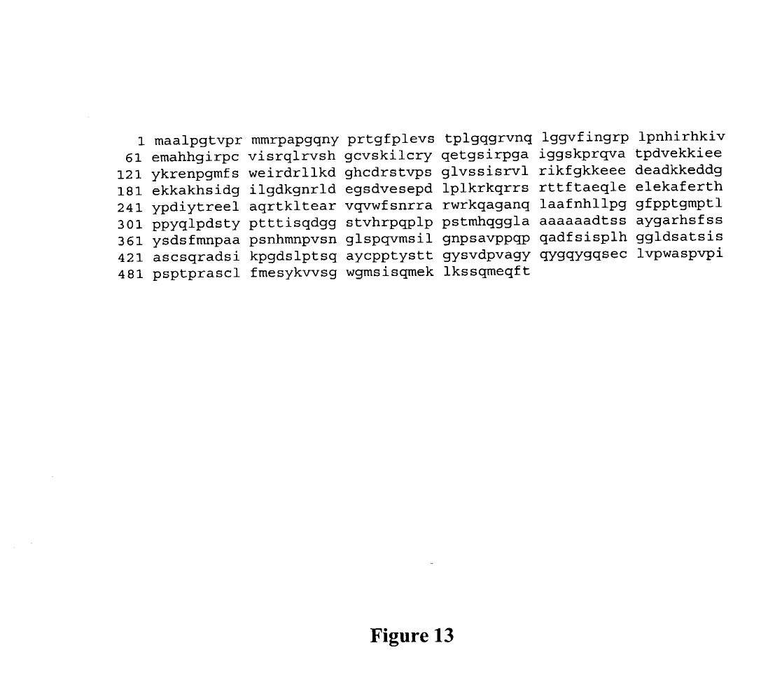

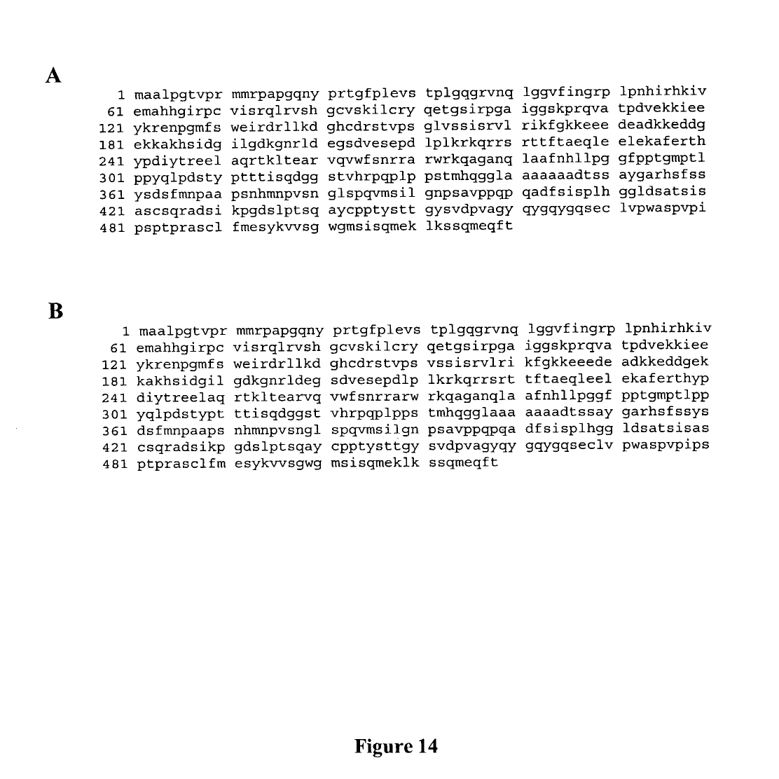

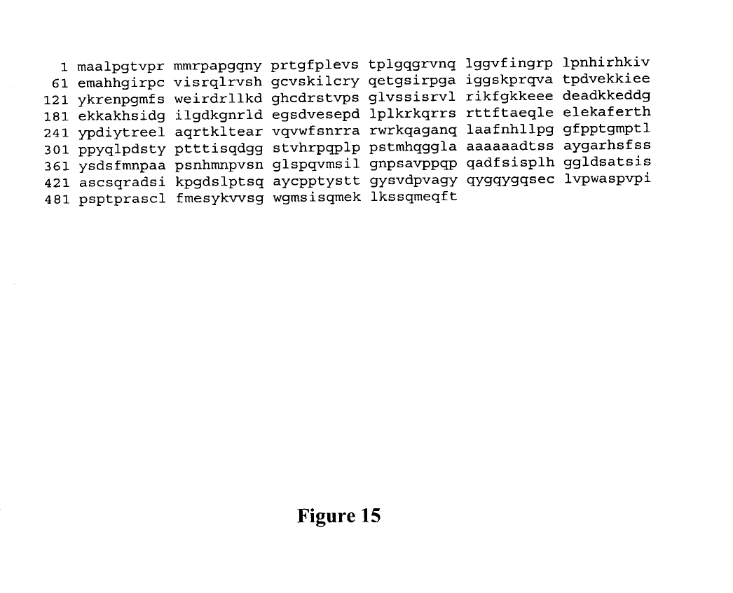

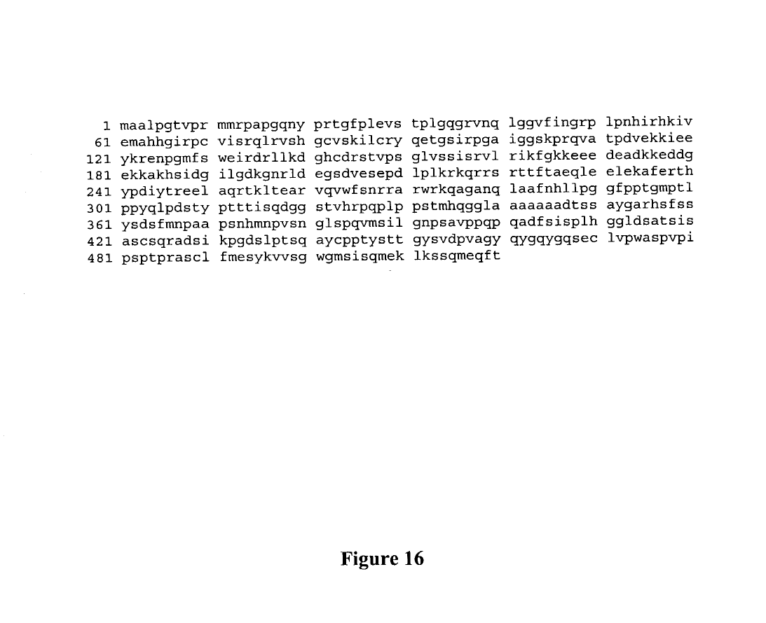

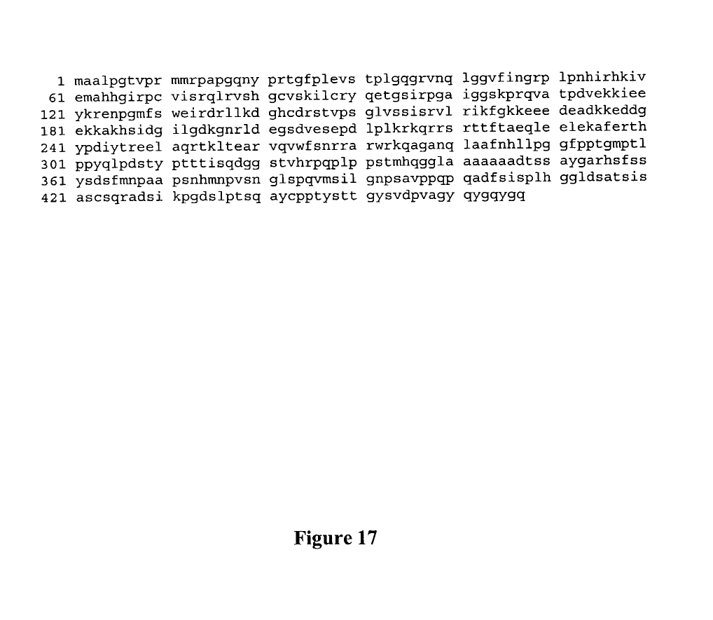

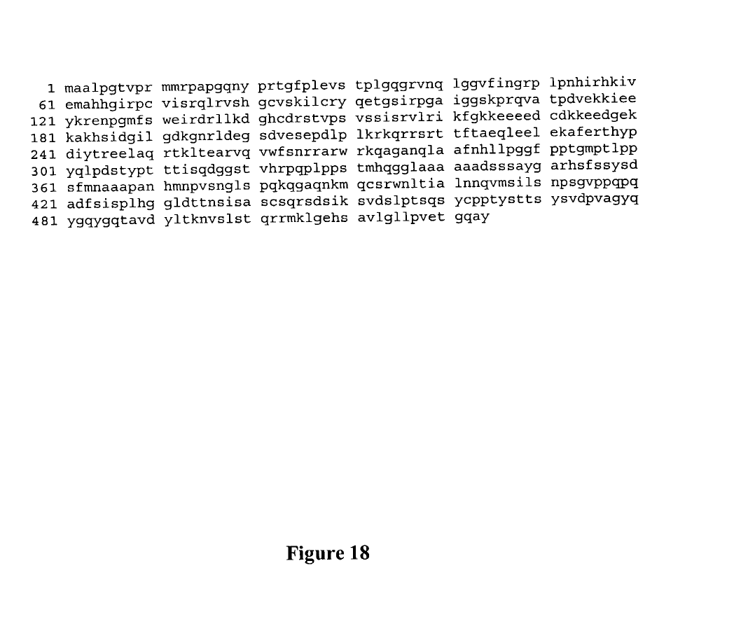

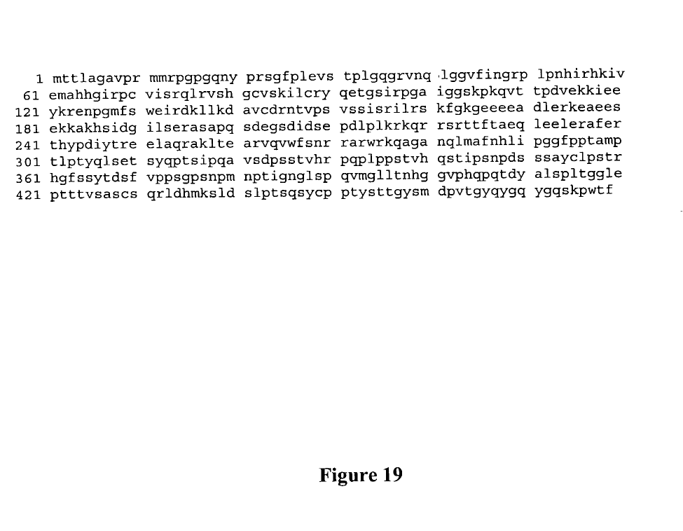

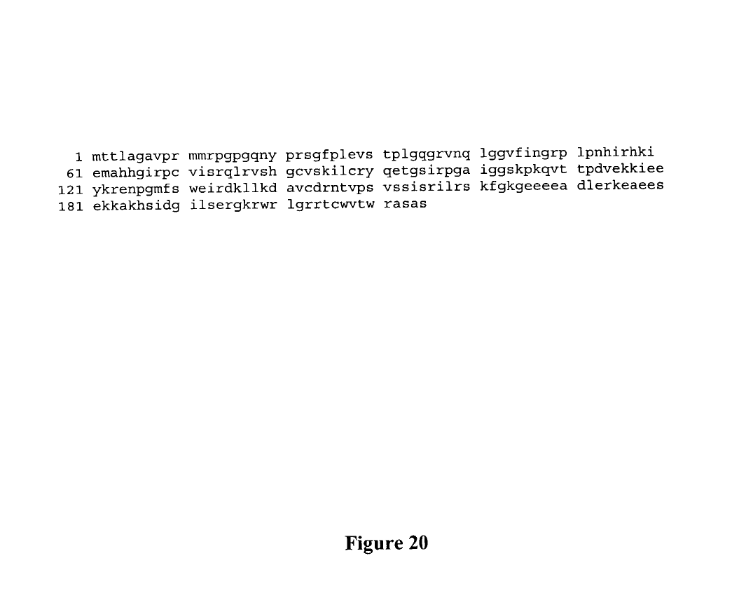

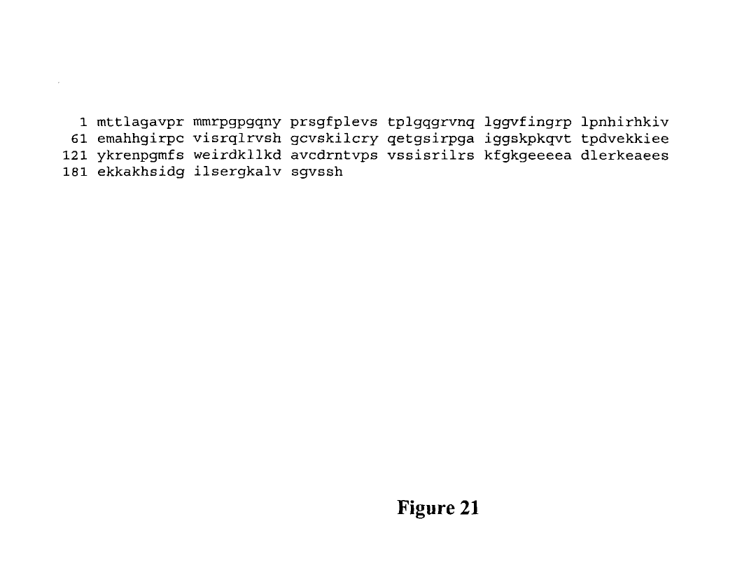

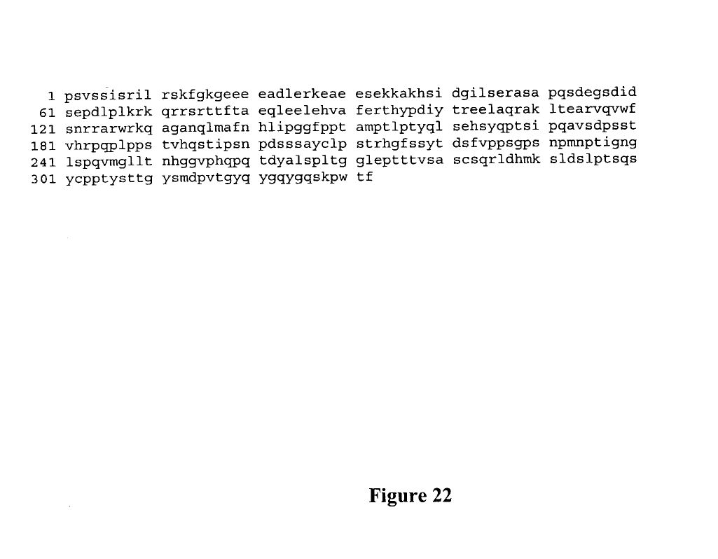

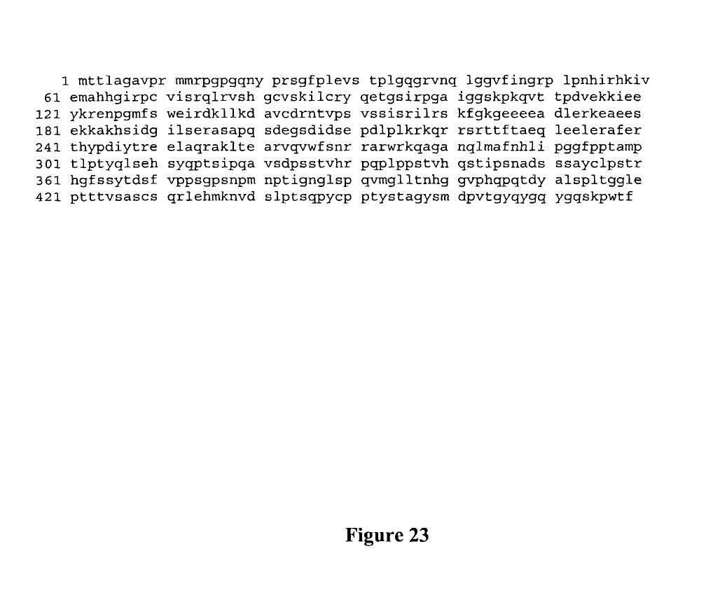

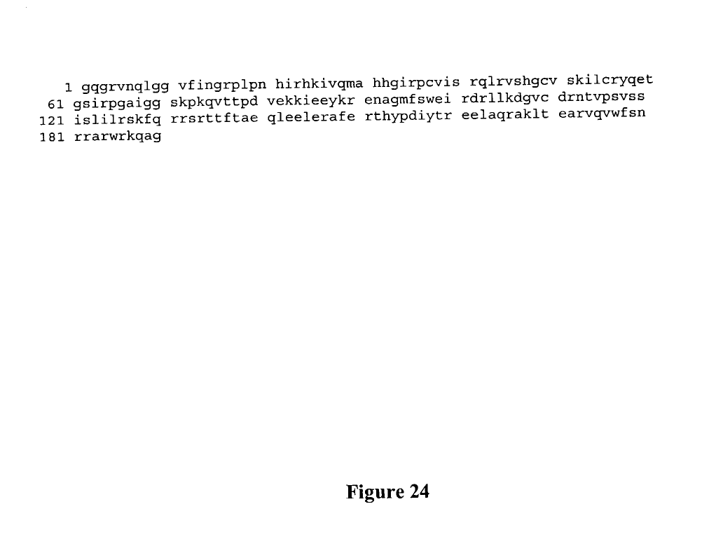

Gene sequences encoding Pax7 and Pax3 are known and a worker skilled in the art would readily appreciate that these sequences can be obtained from publicly available databases, for example, GenBank. For example, NCBI Accession number AL021528 provides the sequence of a human Pax7 gene. Provided herein are non-limiting examples of amino acid sequences that can be expressed by the Pax-encoding vectors of the present invention (see FIGS. 13 through 24).

Nucleic acids comprising a sequence that encodes Pax7, Pax3, or an active variant or fragment thereof can be cloned into a vector using standard techniques that are well known to workers skilled in the art. The Pax-encoding vectors of the present invention facilitate the expression of Pax7, Pax3 or an active variant or fragment thereof such that the expressed protein can induce differentiation of adult pluripotent stem cells. A variety of vectors suitable for use in the preparation of the Pax-encoding vectors of the present invention are known in the art. These vectors must be replicable and viable in the stem cells to be differentiated. The vector used in the preparation of the Pax-encoding vector of the present invention may be, for example, in the form of chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies.

Viral based systems provide the advantage of being able to introduce relatively high levels of a heterologous nucleic acid into a variety of cells. Additionally, such viruses can introduce heterologous DNA into nondividing cells. Suitable viral vectors for preparation of the Pax-encoding vector of the present invention for use in mammalian cells are well known in the art. These viral vectors include, for example, Herpes simplex virus vectors (U.S. Pat. No. 5,501,979), Vaccinia virus vectors (U.S. Pat. No. 5,506,138), Cytomegalovirus vectors (U.S. Pat. No. 5,561,063), Modified Moloney murine leukemia virus vectors (U.S. Pat. No. 5,693,508), adenovirus vectors (U.S. Pat. Nos. 5,700,470 and 5,731,172), adeno-associated virus vectors (U.S. Pat. No. 5,604,090), constitutive and regulatable retrovirus vectors (U.S. Pat. Nos. 4,405,712; 4,650,764 and 5,739,018, respectively), papilloma virus vectors (U.S. Pat. Nos. 5,674,703 and 5,719,054), and the like.

In one embodiment of the present invention, adenovirus-Pax7 vectors are employed to induce specification of stem cells in culture. Any of the Pax-encoding vectors described herein may be employed to induce specification or differentiation of adult pluripotent stem cells.

As used herein, “retroviral vector” refers to the well known gene transfer plasmids that have an expression cassette encoding an heterologous gene residing between two retroviral LTRs. Retroviral vectors typically contain appropriate packaging signals that enable the retroviral vector, or RNA transcribed using the retroviral vector as a template, to be packaged into a viral virion in an appropriate packaging cell line (see, for example, U.S. Pat. No. 4,650,764).

Suitable retroviral vectors for use herein are described, for example, in U.S. Pat. No. 5,252,479, and in WIPO publications WO 92/07573, WO 90/06997, WO 89/05345, WO 92/05266 and WO 92/14829, incorporated herein by reference, which provide a description of methods for efficiently introducing nucleic acids into human cells using such retroviral vectors. Other retroviral vectors include, for example, the MMTV vectors (U.S. Pat. No. 5,646,013), vectors described supra, and the like.

In the preparation of the Pax-encoding vectors of the present invention the nucleic acid sequence encoding the Pax protein is placed under the control of a suitable promoter. Suitable promoters which may be employed include, but are not limited to, adenoviral promoters, such as the adenoviral major late promoter; or hetorologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters. The promoter also may be the native promoter which controls the genes encoding the Pax proteins.

In accordance with one embodiment of the present invention the Pax-encoding vectors may contain additional sequences that encode heterologous biologically active proteins and/or polypeptides. For example, a Pax-encoding vector of the present invention may additionally express a therapeutic protein, such as a growth or trophic factor (e. g., GDNF, neurturin, BDNF, bFGF, NT-3, TGF-P), a transcription factor (e. g., Nurr-1), or an immunosuppressant and operably linked to a suitable promoter. The expression of such a therapeutic protein may be beneficial in order to enhance the survival of cell transplants or increase the therapeutic potential of the cells following transplant. For example, the vectors can be introduced into pluripotent stem cells that are capable of differentiating as muscle cells prior to transplantation into Duschenne patients.

Isolation and Culture of Stem Cells

Methods of cell isolation and culture are described in numerous publications known to the art, for example “Culture of Animal Cells: A Manual of Basic Technique”, 4th Ed. (R. I. Freshney, 2000), and “Current Protocols in Cell Biology” (Wiley & Sons (eds), 2000).

Useful naive stem cells include adult pluripotential stem cells, which may be isolated from bone marrow using conventional methodologies, (see, for example, Faradji et al., (1988) Vox Sang., 55 (3):133-138 or Broxmeyer et al., (1989) PNAS 86:3828-3832), as well as naive stem cells obtained from blood.

Mesenchymal stem cells (MSCs) are the formative pluripotent blast or embryonic-like cells found in bone marrow, blood, dermis, and periosteum that are capable of differentiating into specific types of mesenchymal or connective tissues including adipose, osseous, cartilaginous, elastic, muscular, and fibrous connective tissues (U.S. Pat. No. 5,736,396). The specific differentiation pathway which these cells enter depends upon various influences from mechanical influences and/or endogenous bioactive factors, such as growth factors, cytokines, and/or local microenvironmental conditions established by host tissues. Although these cells are normally present at very low frequencies in bone marrow, a process for isolating, purifying, and mitotically expanding the population of these cells in tissue culture is reported in Caplan et al. U.S. Pat. Nos. 5,197,985 and 5,226,914 and 5,736,396. Factors which have myogenic inductive activity on human MSCs are present in several classes of molecules, especially cytidine analogs, such as 5-azacytidine and 5-aza-2′-deoxycytidine. The effect of these modulating factors on human MSCs is disclosed in Caplan et al. U.S. Pat. No. 5,736,396.

Suitable solid tissue from which cells can be obtained includes any organ or tissue from adult, mammalian tissue. Any mammalian tissue or organ can be used in this invention, including but not limited to those obtained from mice, cattle, sheep, goat, pigs, dogs, rats, rabbits, and primates (including human). Specific examples of suitable solid tissues include skeletal muscle, brain and central nervous system tissue from which neurons and other supporting cells are derived, skin derived from cultured keratinocytes, germ cells or embryonic stem cells or cells from other organs (liver, pancreas, spleen, kidney, thyroid, etc.). Stem cells and progenitor cells isolated from any other solid organ are also amenable candidates for culturing. Stem cells isolated from solid tissues (the exception to solid tissue is whole blood, including blood, plasma and bone marrow) which were previously unidentified in the literature are also within the scope of this invention.

In adult skeletal muscle, the progenitor cell is referred to as a satellite cell. Normally, satellite cells are dormant, but when muscle is traumatized, these cells divide and differentiate, and so are the source of regenerated skeletal muscle. Methods of isolating, identifying, culturing and differentiating satellite cells are well known to those of skill in the art. For example, in U.S. Pat. No. 5,328,695, (1994) Lucas et al. describe a myogenic protein isolate from mammalian (chick) bone that stimulates lineage commitment and differentiation of skeletal muscle stem cells.

It is understood that the initial medium for isolating stems/progenitors, the medium for proliferation of these cells, and the medium for differentiation of these cells can be the same or different. The medium can be supplemented with a variety of growth factors, cytokines, serum, etc. As a general principle, when the goal of culturing is to keep cells dividing, serum is added to the medium in relatively large quantities (10-20% by volume). Specific purified growth factors or cocktails of multiple growth factors can also be added or sometimes used in lieu of serum. As a general principle, when the goal of culturing is to reinforce differentiation, serum with its mitogens is generally limited (about 1-2% by volume). Specific factors or hormones that promote differentiation and/or promote cell cycle arrest can also be used.

Examples of suitable growth factors are basic fibroblast growth factor (bFGF), vascular endothelial growth factor (VEGF), epidermal growth factor (EGF), transforming growth factors (TGF.alpha. and TGF.beta.), platelet derived growth factors (PDGF's), hepatocyte growth factor (HGF), insulin-like growth factor (IGF), insulin, erythropoietin (EPO), and colony stimulating factor (CSF). Examples of suitable hormone medium additives are estrogen, progesterone or glucocorticoids such as dexamethasone. Examples of cytokine medium additives are interferons, interleukins, or tumor necrosis factor-.alpha. (TNF.alpha).

Following differentiation, the specific differentiated cell types are identified by a variety of means including fluorescence activated cell sorting (FACS), protein-conjugated magnetic bead separation, morphologic criteria, specific gene expression patterns (using RT-PCR), or specific antibody staining. The gene products expressed between two or more given differentiated cell types will vary. For example, following differentiation of skeletal muscle satellite cells, the transcription factors myf5, MyoD, myogenin, and mrf4 are expressed. It is understood that developmental pathways often involve more than one step or stage for differentiation and any of these steps or stages may be affected by variations in culture conditions.

Use of the Pax-Encoding Vectors

One embodiment of the present invention provides a method of inducing myogenic differentiation of adult pluripotent stem cells comprising the step of contacting the stem cells with the Pax-encoding vector under conditions that allow expression of the Pax protein, Pax7, Pax3 or an active variant or fragment thereof. This method optionally includes the step of first obtaining and culturing the stem cells from various sources as described herein.

In a related embodiment of the present invention the Pax-encoding vector is used in combination with one or more separate expression vectors that express a molecule that can, for example, aid in the induction of differentiation or improve the therapeutic potential of the myoblasts that are generated.

The differentiated cells that result from the method of the present invention have various uses, including but not limited to their use as a source material for transplantation in the treatment of muscle disease or disorder in animals, including humans. Additionally, the differentiated cells can be used as a research tool and as part of diagnostic assays.

The present invention further relates to a pharmaceutical composition comprising at least one myoblast prepared using the method of the present invention. According to one embodiment, said myoblast comprised in said pharmaceutical composition is encapsulated. Cell encapsulation methodology has been previously described which allows transplantation of encapsulated cells in treatment of Parkinson's disease (Tresco et al., 1992, ASAIO J. 38, 17-23) or Amyotrophic lateral sclerosis (Aebischer et al., 1996, Hum. Gene Ther. 7, 851-860). According to said specific embodiment, cells are encapsulated by compounds which form a microporous membrane, and said encapsulated cells can further be implanted in vivo. Capsules, for example approximately 1 cm in length containing the cells of interest may be prepared employing a hollow microporous membrane fabricated from poly-ether-sulfone (PES) (Akzo Nobel Faser A G, Wuppertal, Germany; D glon et al, 1996, Hum. Gene Ther. 7, 2135-2146). This membrane has a molecular weight cutoff greater than 1,000,000 Da, which permits the free passage of proteins and nutrients between the capsule interior and exterior, while preventing the contact of transplanted cells with host cells. The entrapped cells may be implanted by intradermal, subdermal, intravenous, intramuscular, intranasal, intracerebral, intratracheal, intraarterial, intraperitoneal, intravesical, intrapleural, intracoronary or intratumoral ways.

In a further embodiment, the invention concerns the use of at least one myoblast cell generated, and eventually modified, as described above for the preparation of a composition for administration into a human tissue. In a preferred embodiment the prepared composition in accordance with the use claimed in the present invention is in a form for administration into a vertebrate tissue. These tissues include those of muscle, skin, nose, lung, liver, spleen, bone marrow, thymus, heart, lymph, bone, cartilage, pancreas, kidney, gall bladder, stomach, intestine, testis, ovary, uterus, rectum, nervous system, eye, gland, connective tissue, blood, tumor etc. The administration may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Moreover, myoblast cells are found to migrate from the original site of administration to other sites, particularly injured sites, e.g. degenerating foci. This migration phenomenom permits the treatment of injured sites by injecting myoblasts into the patient in need, particularly in tissue, usually muscle tissue, proximal to the injuries, although injection into the circulation or at a distal site may also be possible. By employing genetically engineered myoblasts one may provide for directed application of products of interest to the injured region. Usually, cell injection will be about 10⁴ to 10⁷ cells (modified or not) per cm³ of muscle tissue to be treated. In this particular case, the composition according to the invention may also comprise a pharmaceutically acceptable injectable carrier. The carrier is preferably isotonic, hypotonic or weakly hypertonic and has a relatively low ionic strength, such as provided by a sucrose solution. It includes any relevant solvent, aqueous or partly aqueous liquid carrier comprising sterile, pyrogen-free water, dispersion media, coatings, and/or equivalents. The pH of the pharmaceutical preparation is suitably adjusted and buffered.

In a further aspect, the invention relates to a diagnostic kit comprising at least one myoblast cell generated according to the invention useful for in vitro assessment of muscular cellular toxicity or damages of candidate or commercially available pharmaceutical molecules (pre-clinical assays) or for in vitro screening of new drugs. In course of said applications, cell lines generated from Duchenne Muscular Dystrophy patient would be preferred. The cultured myoblasts may also serve as a tool to analyse physiopathology of muscular diseases.

Myoblasts prepared using the methods of the present invention can be used for delivery of a muscle protein to the circulation of a mammal. A muscle protein, as used herein, refers to a protein which, when defective or absent in a mammal, is responsible for a particular muscle disease or disorder. Muscle proteins include dystrophin,calpain-3, sarcoglycan complex members (e.g., a-sarcoglycan, P-sarcoglycan, y-sarcoglycan and 5-sarcoglycan) and laminin o: 2-chain. The term circulation is meant to refer to blood circulation. The term blood refers to the “circulating tissue” of the body, the fluid and its suspended formed elements that are circulated through the heart, arteries, capillaries and veins.

In the method for delivery of a muscle protein to the circulation of a mammal, an effective amount of purified donor myoblasts is transplanted into a mammal in need of such treatment (also referred to as a recipient or a recipient mammal). As used herein, “donor” refers to a mammal that is the natural source of the stem cells that are transformed using the viral vectors of the present invention into myoblasts. Preferably, the donor is a healthy mammal (e.g., a mammal that is not suffering from a muscle disease or disorder). In a particular embodiment, the donor and recipient are matched for immunocompatibility.

Preferably, the donor and the recipient are matched for their compatibility for the major histocompatibility complex (MHC) (human leukocyte antigen (HLA))-class I (e. g., loci A, B, C) and-class II (e. g., loci DR, DQ, DRW) antigens.

Immunocompatibility between donor and recipient are determined according to methods generally known in the art (see, e. g., Charron, D. J., Curr. Opin. Hematol., 3: 416-422 (1996); Goldman, J., Curr. Opin. Hematol., 5: 417-418 (1998); and Boisjoly, H. M. et al., Opthalmology, 93: 1290-1297 (1986)). In an embodiment of particular interest, the recipient a human patient.

As used herein, muscle diseases and disorders include, but are not limited to, recessive or inherited myopathies, such as, but not limited to, muscular dystrophies.

Muscular dystrophies are genetic diseases characterized by progressive weakness and degeneration of the skeletal or voluntary muscles which control movement. The muscles of the heart and some other involuntary muscles are also affected in some forms of muscular dystrophy. The histologic picture shows variation in fiber size, muscle cell necrosis and regeneration, and often proliferation of connective and adipose tissue. Muscular dystrophies are described in the art and include Duchenne muscular dystrophy (DMD), Becker muscular dystrophy (BMD), myotonic dystrophy (also known as Steinert's disease), limb-girdle muscular dystrophies, facioscapulohumeral muscular dystrophy (FSH), congenital muscular dystrophies, oculopharyngeal muscular dystrophy (OPMD), distal muscular dystrophies and Emery-Dreifuss muscular dystrophy. See, e. g., Hoffman et al., N. Engl. J. Med., 318. 1363-1368 (1988); Bonnemann, C. G. et al., Curr. Opin. Ped., 8: 569-582 (1996); Worton, R., Science, 270: 755-756 (1995); Funakoshi, M. et al., Neuromuscul. Disord., 9 (2): 108-114 (1999); Lim, L. E. and Campbell, K. P., Cure. Opin. Neurol., 11 (5): 443-452 (1998); Voit, T., Brain Dev., 20 (2): 65-74 (1998); Brown, R. H., Annu. Rev. Med., 48: 457-466 (1997); Fisher, J. and Upadhyaya, M., Neuromuscul. Disord., 7 (1): 55-62 (1997).

Two major types of muscular dystrophy, DMD and BMD, are allelic, lethal degenerative muscle diseases. DMD results from mutations in the dystrophin gene on the X-chromosome (Hoffman et al., N. Engl. J. Med., 318. 1363-1368 (1988)), which usually result in the absence of dystrophin, a cytoskeletal protein in skeletal and cardiac muscle. BMD is the result of mutations in the same gene (Hoffman et al., N. Engl. J. Med., 318: 1363-1368 (1988)), but dystrophin is usually expressed in muscle but at a reduced level and/or as a shorter, internally deleted form, resulting in a milder phenotype.

Thus, the present invention also provides a method of treating a muscle disease or disorder in a mammal in need thereof comprising administering an effective amount of myoblasts to the mammal. In a particular embodiment, the invention relates to a method of treating a muscular dystrophy in a mammal in need thereof comprising administering an effective amount of myoblasts to the mammal. In another embodiment, the invention relates to a method of treating DMD in a mammal in need thereof comprising administering an effective amount of myoblasts to the mammal. In a third embodiment, the invention relates to a method of treating BMD in a mammal in need thereof comprising administering an effective amount of myoblasts to the mammal. In the latter two embodiments, a proportion of the administered myoblasts can fuse with DMD or BMD host muscle fibres, contributing dystrophin-competent myonuclei to the host fibres (mosaic fibres). The expression of normal dystrophin genes in such fibres can generate sufficient dystrophin in some segments to confer a normal phenotype to these muscle fibre segments.

The invention also relates to a method of treating a limb-girdle muscular dystrophy in a mammal in need thereof comprising administering an effective amount of myoblasts to the mammal.

Myoblasts prepared in accordance with the methods of the present invention can also be used in gene therapy, a utility enhanced by the ability of the myoblasts to proliferate and fuse. Myoblasts can be genetically altered by one of several means known in the art to comprise functional genes which may be defective or lacking in a mammal requiring such therapy. The recombinant myoblasts can then be transferred to a mammal, wherein they will multiply and fuse and, additionally, express recombinant genes. Using this technique, a missing or defective gene in a mammal's muscular system can be supplemented or replaced by infusion of genetically altered myoblasts. Gene therapy using myoblasts can also be applied in providing essential gene products through secretion from muscle tissue to the bloodstream (circulation). Because myoblasts are capable of contributing progeny comprising recombinant genes to multiple, multinucleated myofibres during the course of normal muscular development.

To gain a better understanding of the invention described herein, the following examples are set forth. It should be understood that these examples are for illustrative purposes only. Therefore, they should not limit the scope of this invention in any way.

### EXAMPLES

Materials and Methods

Molecular Cloning of Pax7 and Expression Analysis

RDA was performed as described by Hubank and Schatz, 1994. Satellite cell derived myoblast cDNA was subtracted twice against mouse embryonic fibroblast (MEF) cDNA (1:100; 1:400) and once against skeletal muscle cDNA (1:400) to generate the final difference products. The full-length mouse cDNA for Pax7 was isolated by screening an adult mouse skeletal muscle library (Clontech) using the RDA clone as a probe (Maniatis et al., 1982).

Total RNA was extracted as previously described (Chomczynski and Sacchi, 1987). Northern Analysis of 20 μg of total RNA from tissue or cell cultures was performed as per Maniatis et al., 1982. In situ hybridisation for Pax7 mRNA was performed as described elsewhere (Braissant and Wahli, 1998). Sections were counter-stained with 100 μg/mL Propidium Iodide (Sigma) in PBS for 10 minutes at room temperature. Three different Pax7 sequences from the full-length cDNA were used as cRNA probes: Pax7-Sal1: nts 150-1600; dp3-7 nts 4200-4700; Pax7-Cla1: nts 515-1500.

Myoblast and Stem Cell Culture

Primary muscle cultures were isolated as per Sabourin et al., 1999. Primary MEFs were isolated from 13.5-day-old Balb/c mouse embryos (Robertson, 1987). Single muscle fibers were isolated from hind limb skeletal muscles as described previously (Cornelison and Wold, 1997). Individual fibers were cultured in methocult GF M3434 containing 15% FBS, 1% BSA, 10⁻⁴M 2-Mercaptoethanol, 10 μg/mL pancreatic insulin, 200 μg/mL Transferrin, 50 ng/mL SCF, 10 ng/mL IL-3, 10 ng/mL IL-6 and 3 units/mL EPO (Stem Cell Technologies) for 48 hr-10 days.

For hematopoietic colony forming assays, cell suspensions were derived from skeletal muscle by digestion in 0.4% collagenase Type A (Roche)/DMEM for 1.5 hr at 37° C., filtered (74 μm Costar Netwell) and resuspended at 100 cells/μl in 10% horse serum/DMEM. 10,000 cells were cultured in 3 mL of methocult (Stem Cell Technologies) for 14 days.

Fluorescence Activated Cell Sorting (FACS)

Hoechst staining and FACS analysis was essentially performed as described previously (Goodell et al., 1996). FACS was performed on a Becton-Dickinson FacStar flow cytometer equipped with dual lasers. Hoechst dye was excited at 350 nm and its fluorescence was measured at two wavelengths using a 424BP44 filter (Blue emission) and a 650LP filter (Red emission). A 640 DMSP mirror was used to separate wavelengths.

Immunocytochemistry and Electron Microscopy

Primary cell cultures or colonies picked from methocult medium were fixed and stained as described elsewhere (Sabourin et al., 1999) using anti-c-Met SP260 (Santa Cruz); anti-desmin DE-U-10 (DAKO), anti-mouse Ly-6G (clone RB6-8C5) (Pharmingen); anti-mouse Integrin α_{M} (M1/70) (Pharmingen) and MF20 mAb (anti-Myosin Heavy Chain).

Gastrocnemius muscle was prepared for transmission electron microscopy by overnight fixation at 4° C. in 2% gluteraldehyde/0.1 M Cacodylate (pH 7.4) and processed using standard procedures as described elsewhere (Kablar, 1995). Randomly chosen fields were viewed with a Jeol 1200EX Biosystem TEM. Diaphragm and tibialis anterior muscles were prepared for HE staining as described elsewhere (Bancroft and Stevens, 1990).

### Example I

Identification of Genes Expressed in Satellite Cell Derived Myoblasts

Muscle satellite cells represent a distinct lineage of myogenic progenitors responsible for the postnatal growth, repair and maintenance of skeletal muscle (reviewed by Seale and Rudnicki, 2000). At birth, satellite cells account for about 30% of sublaminar muscle nuclei in mice followed by a decrease to less than 5% in a 2 month old adult (Bischoff, 1994). This decline in satellite cell nuclei reflects the fusion of satellite cells during the postnatal growth of skeletal muscle (Gibson and Schultz, 1983). Satellite cells were originally defined on the basis of their unique position in mature skeletal muscle and are closely juxtaposed to the surface of myofibers such that the basal lamina surrounding the satellite cell and its associated myofiber is continuous (Bischoff, 1994).

In mice over 2 months of age, satellite cells in resting skeletal muscle are mitotically quiescent and are activated in response to diverse stimuli including stretching, exercise, injury, and electrical stimulation (Appell et al., 1988; Rosenblatt et al., 1994; Schultz et al., 1985; reviewed by Bischoff, 1994). The descendents of activated satellite cells, called myogenic precursor cells (mpc), undergo multiple rounds of cell division prior to fusion with new or existing myofibers. The total number of quiescent satellite cells in adult muscle remains constant over repeated cycles of degeneration and regeneration, suggesting that the steady state satellite cell population is maintained by self-renewal (Gibson and Schultz, 1983; Schultz and Jaryszak, 1985; Morlet et al., 1989). Therefore, satellite cells have been suggested to form a population of monopotential stem cells that are distinct from their daughter myogenic precursor cells as defined by biological and biochemical criteria (Bischoff, 1994; Grounds and Yablonka-Reuveni, 1993).

Satellite cells clearly represent the progenitors of the myogenic cells that give rise to the majority of the nuclei within adult skeletal muscle. However recent studies have identified a population of pluripotential stem cells, also called side-population (SP) cells in adult skeletal muscle. Muscle-derived SP cells are readily isolated by fluorescence activated cell sorting (FACS) on the basis of Hoechst dye exclusion (Gussoni et al., 1999; Jackson et al., 1999). Purified SP cells derived from muscle exhibit the capacity to differentiate into all major blood lineages following tail vein injection into lethally irradiated mice (Jackson et al., 1999). Of particular significance is the observation that transplanted SP cells isolated from bone marrow or muscle actively participate in myogenic regeneration. However only muscle-derived SP cells appear to give rise to myogenic satellite cells (Gussoni et al., 1999). In addition, SP cells convert to desmin-expressing myoblasts following exposure to appropriate cell culture conditions (Gussoni et al., 1999). However, whether SP cells are equivalent to satellite cells, are progenitors for satellite cells or alternatively represent an entirely independent cell population has remained unclear.

The gene expression profile of quiescent satellite cells and their activated progeny is largely unknown. Quiescent satellite cells express the c-met receptor (receptor for HGF) and M-cadherin protein (Cornelison and Wold, 1997; Irintchev et al., 1994). Activated satellite cells up regulate MyoD or Myf5 prior to entering S-phase (Cornelison and Wold, 1997). Proliferating myogenic precursor cells, the daughter cells of satellite cells, express desmin, Myf5, MyoD and other myoblast specific markers (Cornelison and Wold, 1997; George-Weinstein et al., 1993). Nevertheless, the paucity of cell-lineage specific markers has been a significant impediment to understanding the relationship between satellite cells and their progeny.

Based on our poor understanding of molecular events responsible for satellite cell development and activation, a PCR based subtractive hybridisation approach (Hubank and Schatz, 1994) was used to identify tissue-specific genes expressed in the satellite cell myogenic lineage. Results from this analysis identified several myoblast-specific genes potentially involved in satellite cell function. Pax7 was selected for further analysis based on the established role of the closely related Pax3 protein in regulating the developmental program of embryonic myoblasts (Tajbakhsh et al., 1997; Maroto et al., 1997).

To gain insight into the developmental program responsible for the differentiation and activation of skeletal muscle satellite cells, representational difference analysis of cDNAs (RDA) (Hubank and Schatz, 1994) was employed to identify genes expressed specifically in satellite cell derived myoblasts. This analysis resulted in the identification of 17 distinct products corresponding to 12 known and 5 potentially novel genes by searching GenBank (NCBI) using the FASTA program (unpublished). RDA clone dp3-7 encoded a fragment from within the Pax7 mRNA. Pax7 is a member of the paired-box family of transcription factors that play important regulatory roles in the development of diverse cell lineages (Mansouri, 1999). Therefore, a full-length 4.3-kb Pax7 cDNA was isolated from an adult mouse skeletal muscle cDNA library (Clontech) to facilitate further analyses (NCBI Accession Number: AF254422).

### Example II

Pax7 is Specifically Expressed in Proliferating Myoblasts

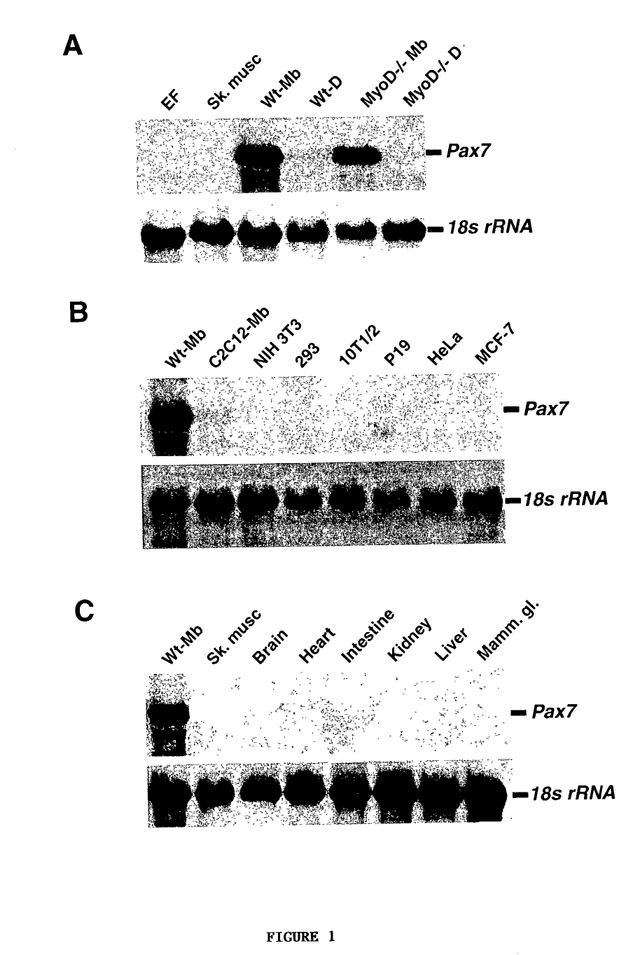

Detailed expression analysis of the distribution of Pax7 mRNA was conducted by Northern analysis (FIG. 1). These analyses demonstrated that Pax7 was expressed exclusively in proliferating primary myoblasts, with comparable levels of expression in both wild type and MyoD^{-/-} cultures (FIG. 1A). However, Pax7 mRNA was down regulated following myogenic differentiation (FIG. 1A). Furthermore, Pax7 was not expressed at detectable levels in a variety of non-muscle cell lines (FIG. 1B). Rather, Pax7 was strictly expressed in myogenic cells including low levels in proliferating C2C12 mouse myoblasts, which are a continuous cell line originally derived from satellite cells (FIG. 1B). In addition, Pax7 mRNA was not detectable in 20 μg of total RNA from several adult mouse tissue samples (FIG. 1C). Analysis of polyA⁺ RNA from select mouse tissues revealed expression of Pax7 at low levels only in adult skeletal muscle (not shown). Therefore, in adult mice Pax7 expression appears specific to the satellite cell myogenic-lineage.

### Example III

Pax7 is Expressed in Satellite Cells

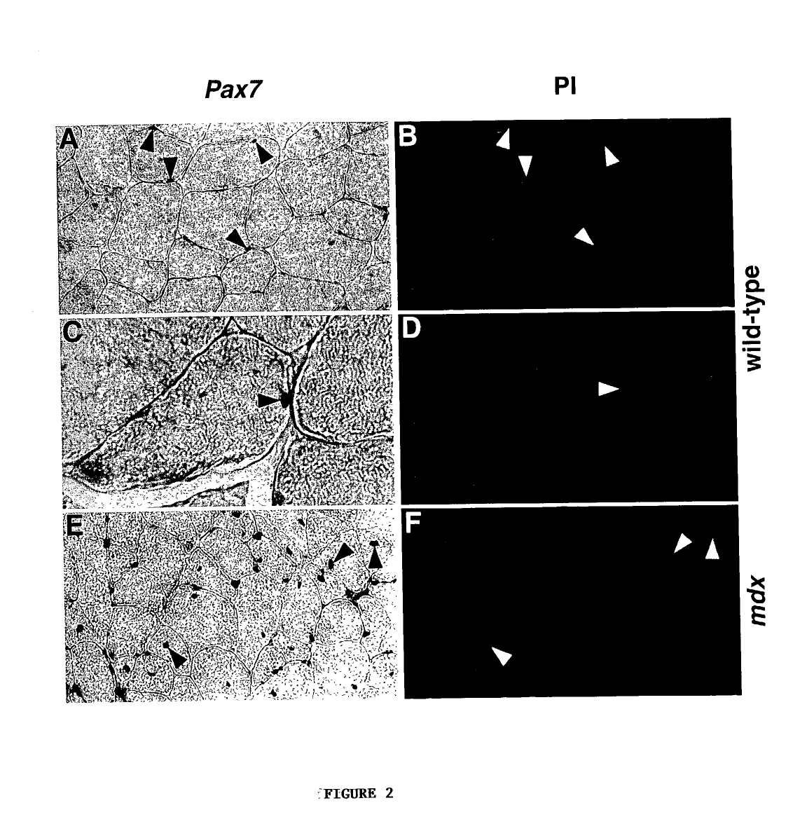

To localise Pax7 mRNA in skeletal muscle, in situ hybridisation was performed on fresh frozen sections of tibialis anterior and gastrocnemius muscles from wild type (Balb/c), MyoD^{-/-}, mdx and compound mutant mdxMyoD^{-/-} animals. Interestingly, Pax7 mRNA was associated with a subset of nuclei in discrete peripheral locations within undamaged wild type (wt) (FIGS. 2A,C) and MyoD^{-/-} (not shown) skeletal muscle. Propidium-Iodide (PI) staining was used to identify all nuclei within skeletal muscle thereby allowing for the enumeration of Pax7 positive cells (FIGS. 2B,D,F). The in situ hybridization was repeated on muscle sections from three independent mice using three separate sequences as anti-sense cRNA probes to verify the expression patterns described. Approximately 5% of muscle nuclei (including satellite cell nuclei and myonuclei) were associated with Pax7 expression in adult wild type muscle. By contrast, the number of Pax7 positive cells increased to 22% in MyoD^{-/-} muscle. The increased expression of Pax7 in MyoD^{-/-} muscle strongly supports the notion that Pax7 is expressed in satellite cells as previous work has revealed that MyoD-deficient muscle contains increased numbers of satellite cells (Megeney et al., 1996). At high magnification (200×), Pax7 appeared to be expressed in cells residing beneath the basal lamina of wild type muscle fibers in positions characteristic for quiescent satellite cells (FIG. 2C).

To determine whether Pax7 was up regulated in regenerating skeletal muscle, 3-week-old mdx and compound mutant mdxMyoD^{-/-} skeletal muscle was analyzed by in situ hybridization. Due to lack of dystrophin protein, mdx muscle undergoes repeated cycles of muscle degeneration and regeneration (Sicinski et al., 1989). As predicted, based on high levels of expression in cultured satellite cell derived myoblasts, Pax7 was widely expressed in regenerating areas of mdx and mdxMyoD^{-/-} skeletal muscle (FIG. 2E). Centrally located nuclei within muscle fibers of mdx (FIG. 2E), MyoD^{-/-} (not shown) and mdxMyoD^{-/-} (not shown) muscle were also associated with Pax7 expression, suggesting that recently activated and fusing myogenic precursors express Pax7. Lastly, a similar distribution of immunoreactive nuclei was observed in muscle sections stained with anti-Pax7 antibody (Developmental Studies Hybridoma Bank). Taken together, the expression analysis supports the notion that Pax7 is expressed within the satellite cell lineage. Therefore, these results raise the hypothesis that Pax7 is required for the ontogeny or function of muscle satellite cells.

### Example IV

Skeletal Muscle Deficiency in Pax7 Mutant Animals

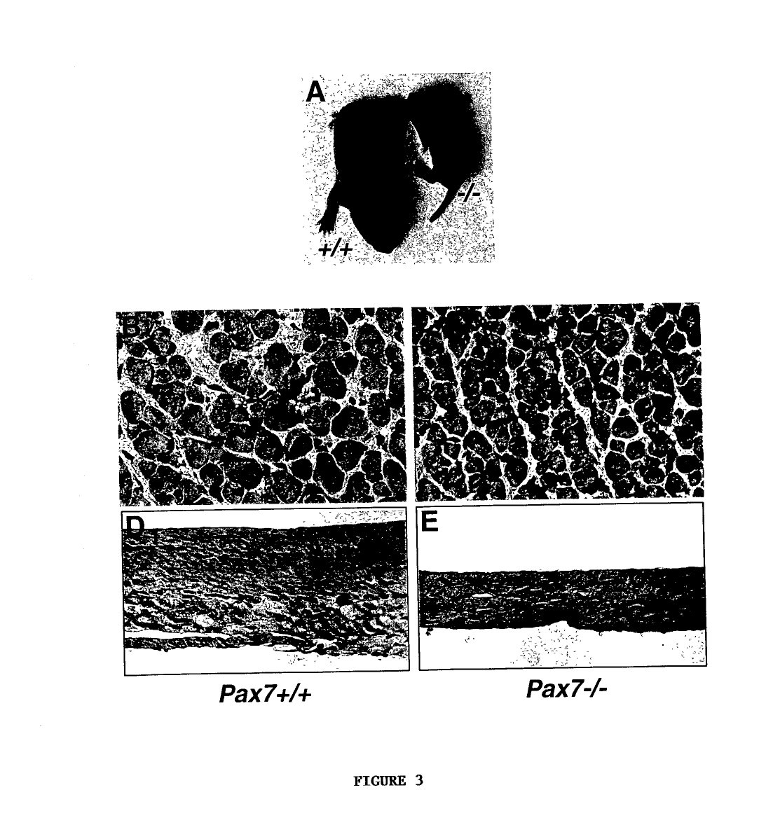

To evaluate possible roles for Pax7 in the formation or function of satellite cells, we examined skeletal muscle from mice carrying a targeted null mutation in Pax7 (Mansouri et al., 1996). Mice deficient for Pax7 express muscle-specific markers including MyoD and Myf5 in a normal spatial and temporal pattern within the developing myotome (Mansouri et al., 1996). However, Pax7^{-/-} mice were significantly smaller than their wild type and heterozygous counterparts (FIG. 3A). The body weight of Pax7^{-/-} mice at 7 days of age was 50% reduced in comparison to wild type littermates (N=20). This weight differential increased with age such that at two weeks of age, mutant animals were about 33% the weight of wild type littermates. As previously reported, Pax7 mutant animals failed to thrive and usually died within two weeks after birth (Mansouri et al., 1996). In addition, we observed that mutant mice exhibited muscle weakness characterized by an abnormal gait and splayed hind limbs (not shown). Light microscopic analysis of hematoxylin-eosin (HE) stained lower hind limb skeletal muscle (below the knee) of one-week-old wild type (FIG. 3B) and Pax7^{-/-} (FIG. 3C) animals revealed a 1.5-fold reduced diameter of Pax7 mutant fibres (N=100 fibres). However, the overall organisation of muscle fibres was not affected. Moreover, the diaphragm from 7-day-old Pax7^{-/-} mice (FIG. 3E) was notably thinner than that from their wild type littermates (FIG. 3D). Therefore, the markedly decreased muscle mass and reduced fibre calibre of Pax7 mutant muscle suggested that the postnatal growth phase of skeletal muscle normally mediated by satellite cells was deficient in the absence of Pax7.

### Example V

Absence of Satellite Cell Derived Myoblasts from Pax7^{-/-} Muscle

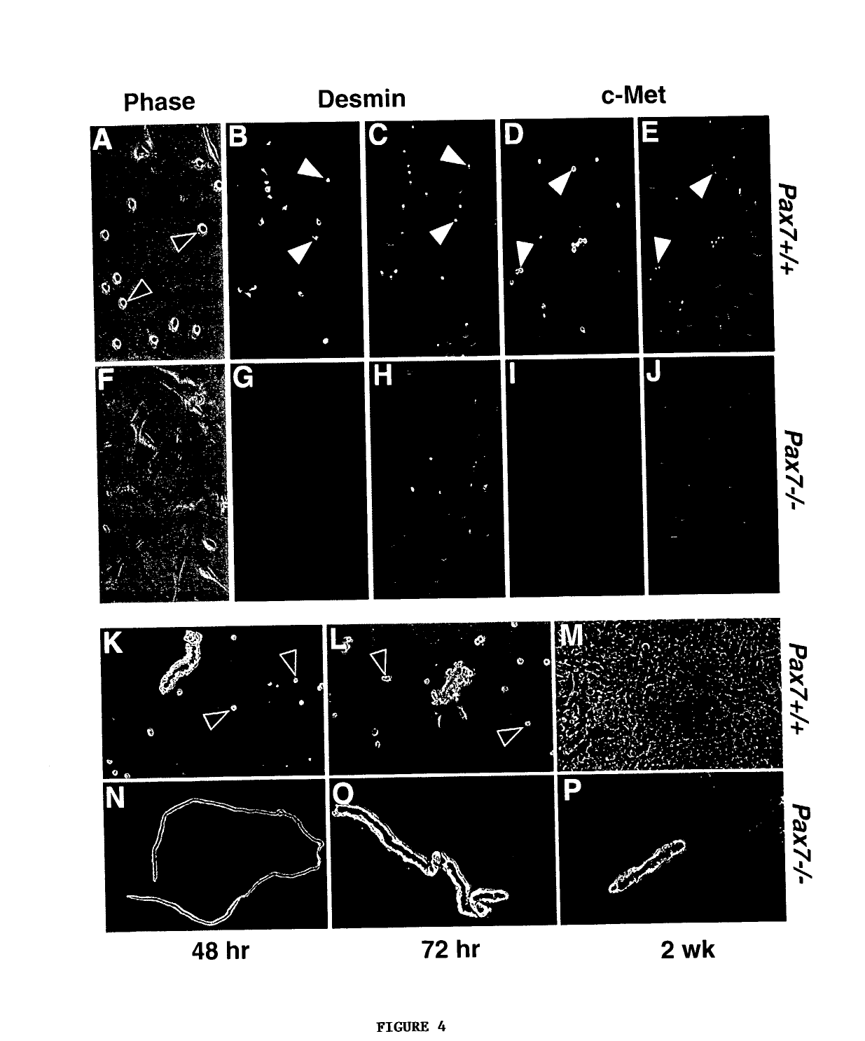

To gain insight into satellite cell function in Pax7 mutant mice, primary cells were cultured directly from the muscle of 7-10 day old wild type mice and Pax7^{-/-} littermates in five independent experiments. After two days in culture, many bursts of satellite cell derived myoblasts were readily identified in wild type primary cultures based on morphological criteria (FIG. 4A) and immunocytochemistry using both anti-desmin and anti-c-Met antibodies that mark satellite cell derived myoblasts (FIGS. 4B-E). Strikingly, no myoblasts were identified in mutant cultures, which instead were uniformly composed of fibroblasts and adipocytes as identified by morphological, and immunochemical criteria (FIGS. 4F-J).

To further investigate whether myogenic cells were present in postnatal Pax7 mutant muscle, individual muscle fibres from 7-10 day old wild type mice and Pax7^{-/-} littermates were isolated in five independent experiments and cultured in methylcellulose stem-cell medium. Methylcellulose stem-cell medium readily promotes the activation, migration and proliferation of satellite cells associated with muscle fibres (Atsushi Asakura and Michael A. Rudnicki, unpublished observation). After 48 and 72 hours in culture, satellite cells associated with wild type fibres generated distinct bursts of desmin-expressing myogenic cells. By contrast, Pax7 mutant muscle fibres did not give rise to any mononuclear cells. Following two weeks in culture, large colonies of fully contractile myosin heavy chain (MHC) expressing myotubes were present in cultures of wild type but not Pax7^{-/-} fibres (not shown). Therefore, these results suggest that satellite cells do not exist, or alternatively fail to proliferate in the absence of Pax7.

### Example VI

Complete Ablation of Satellite Cells in Pax7^{-/-} Muscle

To determine whether or not satellite cells were present in mutant animals, transmission electron microscopy (TEM) was used to analyse skeletal muscle from wild type and Pax7^{-/-} mice. Biopsies from gastrocnemius muscle of three 7-10 day old wild type mice and mutant littermates were analysed by TEM. For each sample, 100 peripheral sublaminar nuclei were analyzed and identified as either satellite cell or myofiber nuclei. Criteria for the identification of satellite cells consisted of: a plasma membrane separating the satellite cell from its adjacent muscle fibre, an overlying basal lamina continuous with the satellite cell and associated fibre, and the characteristic heterochromatic appearance of the nucleus (reviewed in Bischoff, 1994).

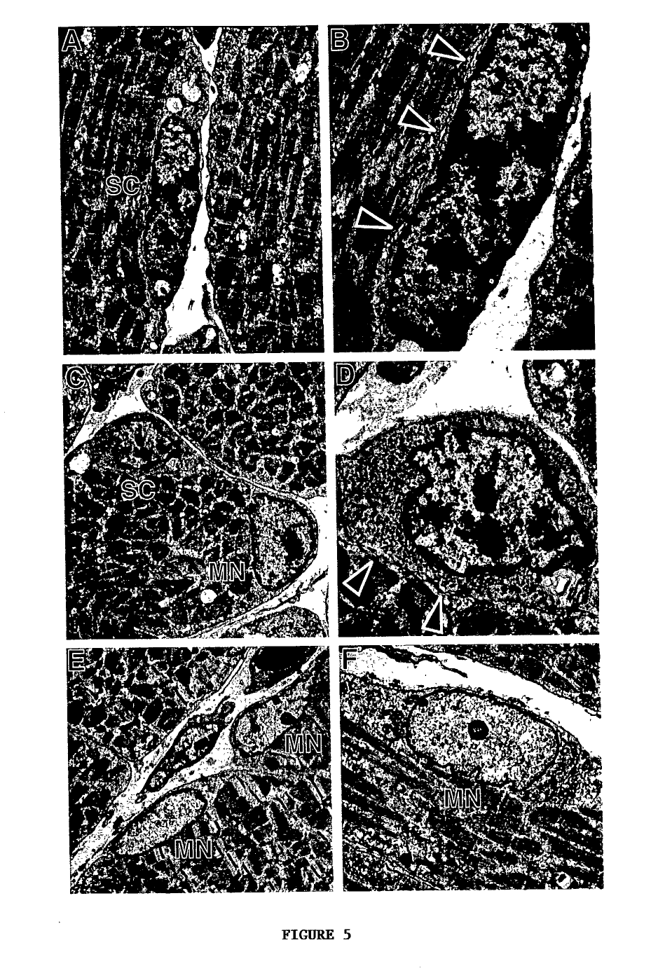

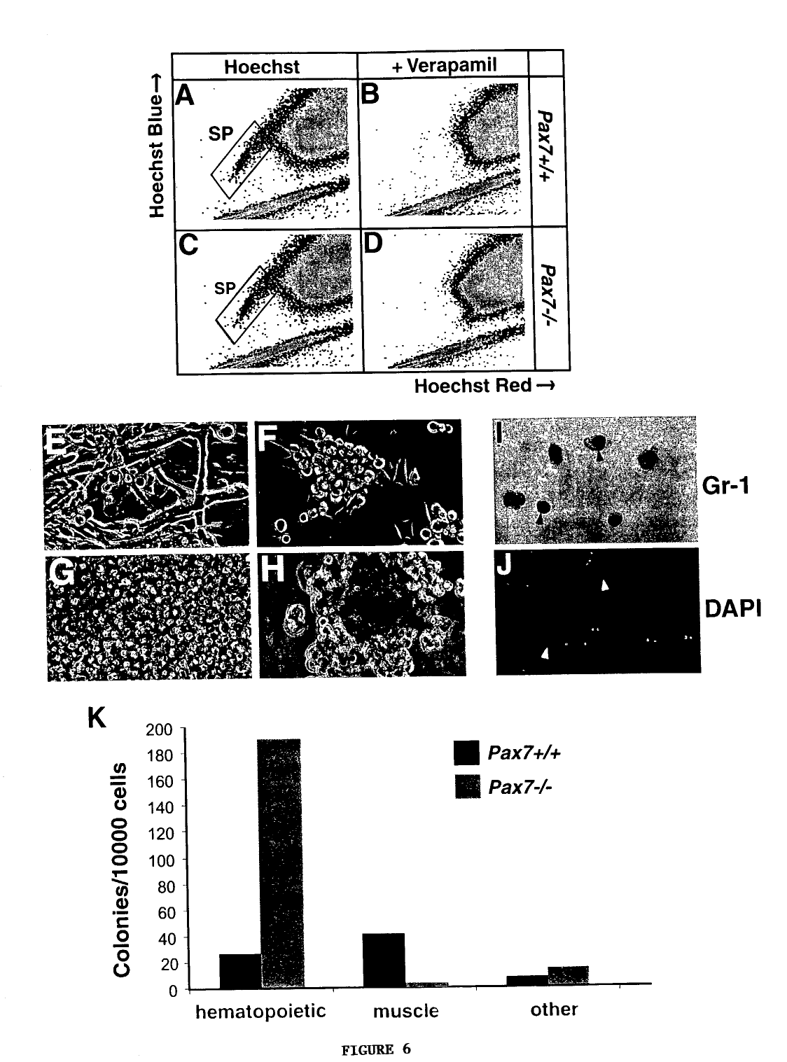

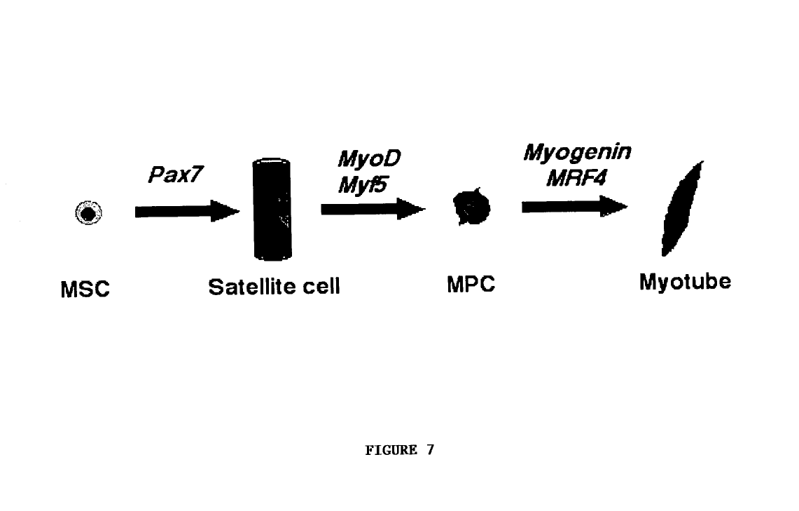

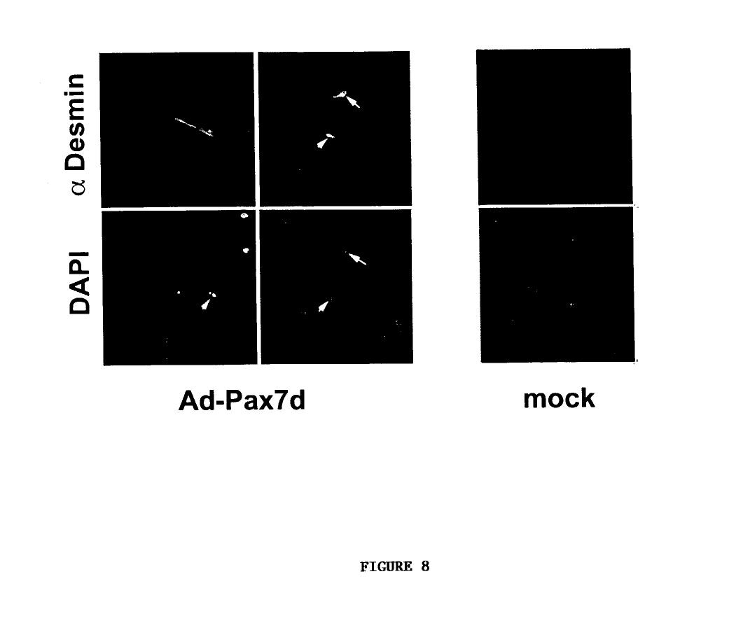

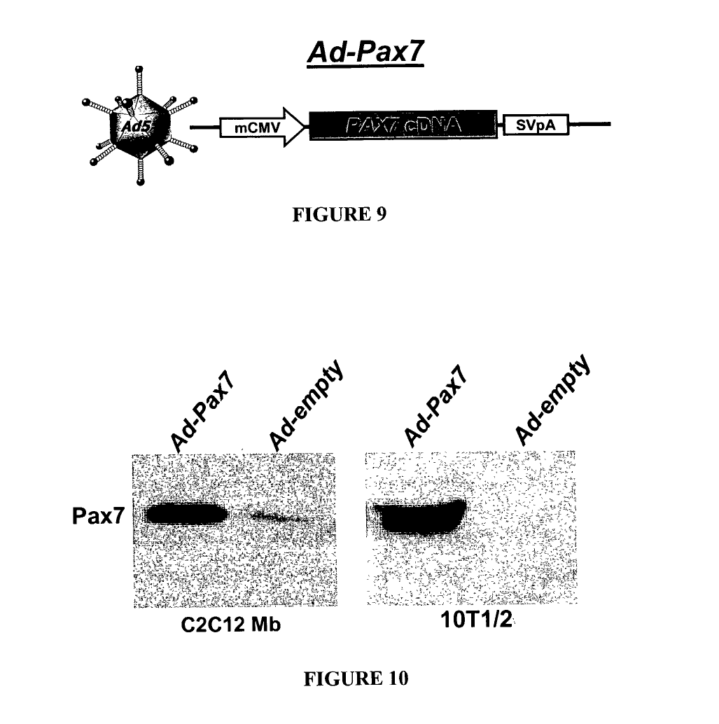

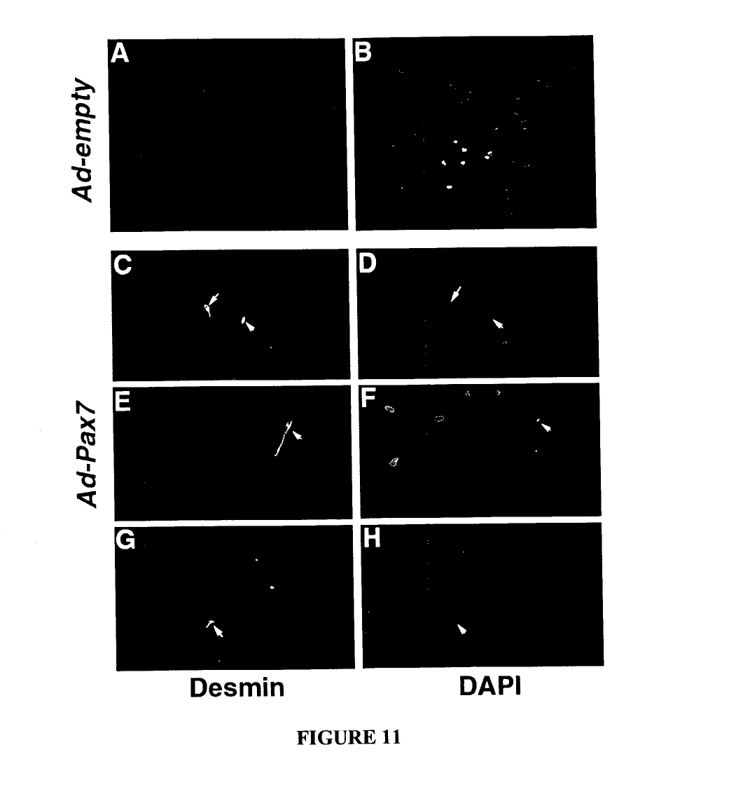

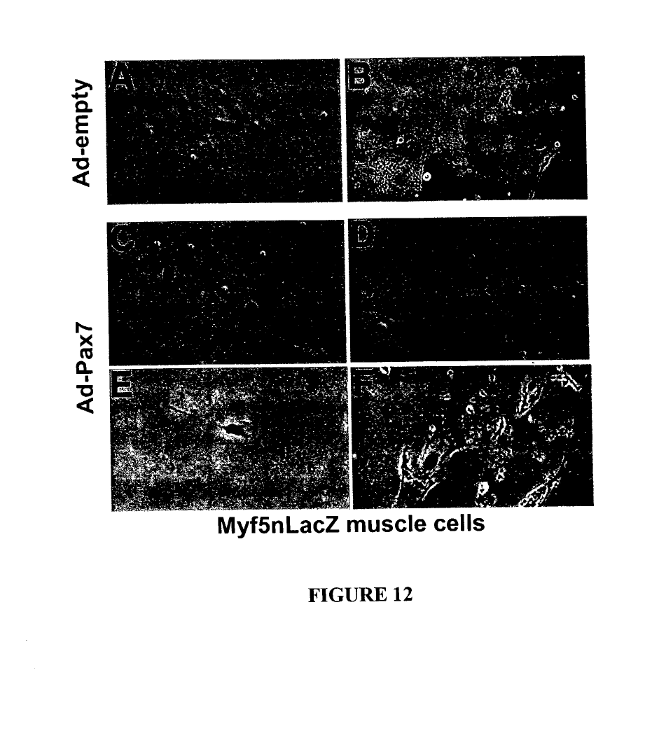

Satellite cells were readily identified in wild type muscle and comprised 25% of peripheral sublaminar nuclei (N=300) (FIGS. 5A-D). By contrast, satellite cells could not be identified in over 300 sublaminar nuclei examined from mutant muscles (FIGS. 5E,F). Furthermore, satellite cells were not found in muscle from E18 embryos (18 days post-coitum) (not shown). Therefore, in the absence of Pax7, complete ablation of muscle satellite cells was observed. The failure of muscle satellite cells to form in Pax7^{-/-} muscle thus unequivocally establishes an essential role for Pax7 in the ontogeny of the satellite cell lineage.

### Example VII

Muscle-Derived SP Cells are Present in Pax7 Mutant Muscle

To investigate the relationship between satellite cells and muscle-derived pluripotent stem cells, fluorescence activated cell sorting (FACS) analysis of cells isolated from wild type and Pax7^{-/-} muscle was performed. Recent work has identified a population of pluripotent stem cells (also called side-population (SP) cells) in skeletal muscle as defined by Hoechst 33342 dye exclusion (Gussoni et al., 1999; Jackson et al., 1999). Cell suspensions isolated directly from one-week-old skeletal muscle were stained with Hoechst dye in the presence or absence

REFERENCES:

Allen, R. E., Sheehan, S. M., Taylor, R. G., Kendall, T. L., and Rice, G. M. (1995). Hepatocyte growth factor activates quiescent skeletal muscle satellite cells in vitro. J Cell Physiol 165, 307-12.

Appell, H. J., Forsberg, S., and Hollmann, W. (1988). Satellite cell activation in human skeletal muscle after training: evidence for muscle fiber neoformation. Int J Sports Med 9, 297-9.

Bancroft, J. D., and Stevens, A. (1990). Theory and practice of histological techniques, 3rd—Edition (Edinburgh; New York: Churchill Livingstone).

Bennicelli, J. L., Advani, S., Schafer, B. W., and Barr, F. G. (1999). PAX3 and PAX7 exhibit conserved cis-acting transcription repression domains and utilize a common gain of function mechanism in alveolar rhabdomyosarcoma. Oncogene 18, 4348-56.

Bischoff, R. (1994). The satellite cell and muscle regeneration. In Myogenesis, A. G. Engel and C. Franszini-Armstrong, eds. (New York: McGraw-Hill), pp. 97-118.

Borycki, A. G., and Emerson, C. P. (1997). Muscle determination: another key player in myogenesis? Curr Biol 7, R620-3.

Braissant, O., and Wahli, W. (1998). Differential expression of peroxisome proliferator-activated receptor-alpha, -beta, and -gamma during rat embryonic development. Endocrinology 139, 2748-54.

Chomczynski, P., and Sacchi, N. (1987). Single-step method of RNA isolation by acid guanidinium thiocyanate- phenol-chloroform extraction. Anal Biochem 162, 156-9.

Cornelison, D. D., and Wold, B. J. (1997). Single-cell analysis of regulatory gene expression in quiescent and activated mouse skeletal muscle satellite cells. Dev Biol 191, 270-83.

Cunningham, B. A., Hemperly, J. J., Murray, B. A., Prediger, E. A., Brackenbury, R., and Edelman, G. M. (1987). Neural cell adhesion molecule: structure, immunoglobulin-like domains, cell surface modulation, and alternative RNA splicing. Science 236, 799-806.

Daston, G., Lamar, E., Olivier, M., and Goulding, M. (1996). Pax-3 is necessary for migration but not differentiation of limb muscle precursors in the mouse. Development 122, 1017-27.

De Angelis, L., Berghella, L., Coletta, M., Lattanzi, L., Zanchi, M., Cusella-De Angelis, M. G., Ponzetto, C., and Cossu, G. (1999). Skeletal myogenic progenitors originating from embryonic dorsal aorta coexpress endothelial and myogenic markers and contribute to postnatal muscle growth and regeneration [see comments]. J Cell Biol 147, 869-78.

Epstein, J. A., Lam, P., Jepeal, L., Maas, R. L., and Shapiro, D. N. (1995). Pax3 inhibits myogenic differentiation of cultured myoblast cells. J Biol Chem 270, 11719-22.

Epstein, J. A., Shapiro, D. N., Cheng, J., Lam, P. Y., and Maas, R. L. (1996). Pax3 modulates expression of the c-Met receptor during limb muscle development. Proc Natl Acad Sci USA 93, 4213-8.

Fleming, T. J., Fleming, M. L., and Malek, T. R. (1993). Selective expression of Ly-6G on myeloid lineage cells in mouse bone marrow. RB6-8C5 mAb to granulocyte-differentiation antigen (Gr-1) detects members of the Ly-6 family. J Immunol 151, 2399-408.

George-Weinstein, M., Foster, R. F., Gerhart, J. V., and Kaufman, S. J. (1993). In vitro and in vivo expression of alpha 7 integrin and desmin define the primary and secondary myogenic lineages. Dev Biol 156, 209-29.

Gibson, M. C., and Schultz, E. (1983). Age-related differences in absolute numbers of skeletal muscle satellite cells. Muscle Nerve 6, 574-80.

Goodell, M. A., Brose, K., Paradis, G., Conner, A. S., and Mulligan, R. C. (1996). Isolation and functional properties of murine hematopoietic stem cells that are replicating in vivo. J Exp Med 183, 1797-806.

Goodell, M. A., Rosenzweig, M., Kim, H., Marks, D. F., DeMaria, M., Paradis, G., Grupp, S. A., Sieff, C. A., Mulligan, R. C., and Johnson, R. P. (1997). Dye efflux studies suggest that hematopoietic stem cells expressing low or undetectable levels of CD34 antigen exist in multiple species. Nat Med 3, 1337-45.

Goulding, M. D., Chalepakis, G., Deutsch, U., Erselius, J. R., and Gruss, P. (1991). Pax-3, a novel murine DNA binding protein expressed during early neurogenesis. Embo J 10, 1135-47.

Grounds, M. D., and Yablonka-Reuveni, Z. (1993). Molecular and cell biology of skeletal muscle regeneration. Mol Cell Biol Hum Dis Ser 3, 210-56.

Gussoni, E., Soneoka, Y., Strickland, C. D., Buzney, E. A., Khan, M. K., Flint, A. F., Kunkel, L. M., and Mulligan, R. C. (1999). Dystrophin expression in the mdx mouse restored by stem cell transplantation. Nature 401, 390-4.

Heanue, T. A., Reshef, R., Davis, R. J., Mardon, G., Oliver, G., Tomarev, S., Lassar, A. B., and Tabin, C. J. (1999). Synergistic regulation of vertebrate muscle development by Dach2, Eya2, and Six1, homologs of genes required for Drosophila eye formation. Genes Dev 13, 3231-43.

Holst, B. D., Wang, Y., Jones, F. S., and Edelman, G. M. (1997). A binding site for Pax proteins regulates expression of the gene for the neural cell adhesion molecule in the embryonic spinal cord. Proc Natl Acad Sci USA 94, 1465-70.

Hubank, M., and Schatz, D. G. (1994). Identifying differences in mRNA expression by representational difference analysis of cDNA. Nucleic Acids Res 22, 5640-8.

Hurko, O., and Walsh, F. S. (1983). Human fetal muscle-specific antigen is restricted to regenerating myofibers in diseased adult muscle. Neurology 33, 737-43.

Irintchev, A., Zeschnigk, M., Starzinski-Powitz, A., and Wemig, A. (1994). Expression pattern of M-cadherin in normal, denervated, and regenerating mouse muscles. Dev Dyn 199, 326-37.

Jackson, K. A., Mi, T., and Goodell, M. A. (1999). Hematopoietic potential of stem cells isolated from murine skeletal muscle [see comments]. Proc Natl Acad Sci USA 96, 14482-6.

Jostes, B., Walther, C., and Gruss, P. (1990). The murine paired box gene, Pax7, is expressed specifically during the development of the nervous and muscular system. Mech Dev 33, 27-37.

Kablar, B. (1995). Structural study on the appearance of innervation in the stomach of mouse and rat embryos. Tissue Cell 27, 309-15.

Kay, P. H., Harmon, D., Fletcher, S., Robertson, T., Ziman, M., and Papadimitriou, J. M. (1998). Pax7 includes two polymorphic homeoboxes which contain rearrangements associated with differences in the ability to regenerate damaged skeletal muscle in adult mice. Int J Biochem Cell Biol 30, 261-9.

Kay, P. H., Mitchell, C. A., Akkari, A., and Papadimitriou, J. M. (1995). Association of an unusual form of a Pax7-like gene with increased efficiency of skeletal muscle regeneration. Gene 163, 171-7.

Leenen, P. J., de Bruijn, M. F., Voerman, J. S., Campbell, P. A., and van Ewijk, W. (1994). Markers of mouse macrophage development detected by monoclonal antibodies. J Immunol Methods 174, 5-19.

Maniatis, T., Fritsch, E. F., and Sambrook, J. (1982). Molecular cloning: a laboratory manual (Cold Spring Harbor, N.Y.: Cold Spring Harbor Laboratory).

Mansouri, A., Goudreau, G., and Gruss, P. (1999). Pax genes and their role in organogenesis. Cancer Res 59, 1707s-1709s; discussion 1709s-1710s.

Mansouri, A., Hallonet, M., and Gruss, P. (1996). Pax genes and their roles in cell differentiation and development. Curr Opin Cell Biol 8, 851-7.

Mansouri, A., Stoykova, A., Torres, M., and Gruss, P. (1996). Dysgenesis of cephalic neural crest derivatives in Pax7−/− mutant mice. Development 122, 831-8.

Maroto, M., Reshef, R., Munsterberg, A. E., Koester, S., Goulding, M., and Lassar, A. B. (1997). Ectopic Pax-3 activates MyoD and Myf-5 expression in embryonic mesoderm and neural tissue. Cell 89, 139-48.

Megeney, L. A., Kablar, B., Garrett, K., Anderson, J. E., and Rudnicki, M. A. (1996). MyoD is required for myogenic stem cell function in adult skeletal muscle. Genes Dev 10, 1173-83.

Morlet, K., Grounds, M. D., and McGeachie, J. K. (1989). Muscle precursor replication after repeated regeneration of skeletal muscle in mice. Anat Embryol 180, 471-8.

Noll, M. (1993). Evolution and role of Pax genes. Curr Opin Genet Dev 3, 595-605.

Nutt, S. L., Heavey, B., Rolink, A. G., and Busslinger, M. (1999). Commitment to the B-lymphoid lineage depends on the transcription factor Pax5 [see comments]. Nature 401, 556-62.

Relaix, F., and Buckingham, M. (1999). From insect eye to vertebrate muscle: redeployment of a regulatory network. Genes Dev 13, 3171-8.

Robertson, E., J. (1987). Embryo-Derived Stem Cell Lines. In Teratocarcinomas and embryonic stem cells: a practical approach, E. J. Robertson, ed. (Oxford: IRL Press Ltd.), pp. 71-112.

Rolink, A. G., Nutt, S. L., Melchers, F., and Busslinger, M. (1999). Long-term in vivo reconstitution of T-cell development by Pax5-deficient B-cell progenitors [see comments]. Nature 401, 603-6.

Rosenblatt, J. D., Yong, D., and Parry, D. J. (1994). Satellite cell activity is required for hypertrophy of overloaded adult rat muscle. Muscle Nerve 17, 608-13.

Sabourin, L. A., Girgis-Gabardo, A., Seale, P., Asakura, A., and Rudnicki, M. A. (1999). Reduced differentiation potential of primary MyoD−/− myogenic cells derived from adult skeletal muscle. J Cell Biol 144, 631-43.

Schafer, B. W., Czerny, T., Bernasconi, M., Genini, M., and Busslinger, M. (1994). Molecular cloning and characterization of a human PAX-7 cDNA expressed in normal and neoplastic myocytes. Nucleic Acids Ref 22, 4574-82.

Schultz, E., and Jaryszak, D. L. (1985). Effects of skeletal muscle regeneration on the proliferation potential of satellite cells. Mech Ageing Dev 30, 63-72.

Schultz, E., Jaryszak, D. L., and Valliere, C. R. (1985). Response of satellite cells to focal skeletal muscle injury. Muscle Nerve 8, 217-22.

Seale, P., and Rudnicki, M. A. (2000). A new look at the origin, function, and “stem-cell” status of muscle satellite cells. Dev Biol 218, 115-24.

Sicinski, P., Geng, Y., Ryder-Cook, A. S., Barnard, E. A., Darlison, M. G., and Barnard, P. J. (1989). The molecular basis of muscular dystrophy in the mdx mouse: a point mutation. Science 244, 1578-80.

Strachan, T., and Read, A. P. (1994). PAX genes. Curr Opin Genet Dev 4, 427-38.

Tajbakhsh, S., Rocancourt, D., Cossu, G., and Buckingham, M. (1997). Redefining the genetic hierarchies controlling skeletal myogenesis: Pax-3 and Myf-5 act upstream of MyoD. Cell 89, 127-38.

Tremblay, P., Dietrich, S., Mericskay, M., Schubert, F. R., Li, Z., and Paulin, D. (1998). A crucial role for Pax3 in the development of the hypaxial musculature and the long-range migration of muscle precursors. Dev Biol 203, 49-61.

Williams, B. A., and Ordahl, C. P. (1994). Pax-3 expression in segmental mesoderm marks early stages in myogenic cell specification. Development 120, 785-96.

Asakura, A., Lyons, G. E., and Tapscott, S. J. (1995). The regulation of MyoD gene expression: conserved elements mediate expression in embryonic axial muscle. Dev Biol 171, 386-98.

Goodell, M. A., Brose, K., Paradis, G., Conner, A. S., and Mulligan, R. C. (1996). Isolation and functional properties of murine hematopoietic stem cells that are replicating in vivo. J Exp Med 183, 1797-806.

Jackson, K. A., Mi, T., and Goodell, M. A. (1999). Hematopoietic potential of stem cells isolated from murine skeletal muscle [see comments]. Proc Natl Acad Sci USA 96, 14482-6.

Ng, P., Parks, R. J., Cummings, D. T., Evelegh, C. M., Sankar, U., and Graham, F. L. (1999). A high-efficiency Cre/loxP-based system for construction of adenoviral vectors. Hum Gene Ther 10, 2667-72.

Tajbakhsh, S., Bober, E., Babinet, C., Pournin, S., Arnold, H., and Buckingham, M. (1996). Gene targeting the myf-5 locus with nlacZ reveals expression of this myogenic factor in mature skeletal muscle fibres as well as early embryonic muscle. Dev Dyn 206, 291-300.

Bennicelli, J. L., Advani, S., Schafer, B. W., and Barr, F. G. (1999). PAX3 and PAX7 exhibit conserved cis-acting transcription repression domains and utilize a common gain of function mechanism in alveolar rhabdomyosarcoma. Oncogene 18, 4348-56.

Borycki, A. G., and Emerson, C. P. (1997). Muscle determination: another key player in myogenesis? Curr Biol 7, R620-3.

Conway, S. J., Henderson, D. J., Kirby, M. L., Anderson, R. H., and Copp, A. J. (1997). Development of a lethal congenital heart defect in the splotch (Pax3) mutant mouse. Cardiovasc Ref 36, 163-73.

Dahl, E., Koseki, H., and Balling, R. (1997). Pax genes and organogenesis. Bioessays 19, 755-65.

Daston, G., Lamar, E., Olivier, M., and Goulding, M. (1996). Pax-3 is necessary for migration but not differentiation of limb muscle precursors in the mouse. Development 122, 1017-27.

Epstein, J. A., Lam, P., Jepeal, L., Maas, R. L., and Shapiro, D. N. (1995). Pax3 inhibits myogenic differentiation of cultured myoblast cells. J Biol Chem 270, 11719-22.

Goulding, M. D., Chalepakis, G., Deutsch, U., Erselius, J. R., and Gruss, P. (1991). Pax-3, a novel murine DNA binding protein expressed during early neurogenesis. Embo J 10, 1135-47.

Graw, J. (1999). Cataract mutations and lens development. Prog Retin Eye Ref 18, 235-67.

Heanue, T. A., Reshef, R., Davis, R. J., Mardon, G., Oliver, G., Tomarev, S., Lassar, A. B., and Tabin, C. J. (1999). Synergistic regulation of vertebrate muscle development by Dach2, Eya2, and Six1, homologs of genes required for Drosophila eye formation. Genes Dev 13, 3231-43.

Jostes, B., Walther, C., and Gruss, P. (1990). The murine paired box gene, Pax7, is expressed specifically during the development of the nervous and muscular system. Mech Dev 33, 27-37.

Kay, P. H., Harmon, D., Fletcher, S., Robertson, T., Ziman, M., and Papadimitriou, J. M. (1998). Pax7 includes two polymorphic homeoboxes which contain rearrangements associated with differences in the ability to regenerate damaged skeletal muscle in adult mice. Int J Biochem Cell Biol 30, 261-9.

Kay, P. H., Harmon, D., Fletcher, S., Ziman, M., Jacobsen, P. F., and Papadimitriou, J. M. (1997). Variation in the methylation profile and structure of Pax3 and Pax7 among different mouse strains and during expression. Gene 184, 45-53.

Kay, P. H., Mitchell, C. A., Akkari, A., and Papadimitriou, J. M. (1995). Association of an unusual form of a Pax7-like gene with increased efficiency of skeletal muscle regeneration. Gene 163, 171-7.

Kay, P. H., and Ziman, M. R. (1999). Alternate Pax7 paired box transcripts which include a trinucleotide or a hexanucleotide are generated by use of alternate 3′ intronic splice sites which are not utilized in the ancestral homologue. Gene 230, 55-60.

Khan, J., Bittner, M. L., Saal, L. H., Teichmann, U., Azorsa, D. O., Gooden, G. C., Pavan, W. J., Trent, J. M., and Meltzer, P. S. (1999). cDNA microarrays detect activation of a myogenic transcription program by the PAX3-FKHR fusion oncogene. Proc Natl Acad Sci USA 96, 13264-9.

Mansouri, A., Chowdhury, K., and Gruss, P. (1998). Follicular cells of the thyroid gland require Pax8 gene function. Nat Genet 19, 87-90.

Mansouri, A., Goudreau, G., and Gruss, P. (1999). Pax genes and their role in organogenesis. Cancer Res 59, 1707s-1709s; discussion 1709s-1710s.

Mansouri, A., Stoykova, A., and Gruss, P. (1994). Pax genes in development. J Cell Sci Suppl 18, 35-42.

Mansouri, A., Stoykova, A., Torres, M., and Gruss, P. (1996). Dysgenesis of cephalic neural crest derivatives in Pax7−/− mutant mice. Development 122, 831-8.

Maroto, M., Reshef, R., Munsterberg, A. E., Koester, S., Goulding, M., and Lassar, A. B. (1997). Ectopic Pax-3 activates MyoD and Myf-5 expression in embryonic mesoderm and neural tissue. Cell 89, 139-48.

Naldini, L., Blomer, U., Gallay, P., Ory, D., Mulligan, R., Gage, F. H., Verma, I. M., and Trono, D. (1996). In vivo gene delivery and stable transduction of nondividing cells by a lentiviral vector. Science 272, 263-7.

Noll, M. (1993). Evolution and role of Pax genes. Curr Opin Genet Dev 3, 595-605.

Nutt, S. L., Thevenin, C., and Busslinger, M. (1997). Essential functions of Pax-5 (BSAP) in pro-B cell development. Immunobiology 198, 227-35.

Peters, H., Wilm, B., Sakai, N., Imai, K., Maas, R., and Balling, R. (1999). Pax1 and Pax9 synergistically regulate vertebral column development. Development 126, 5399-408.

Relaix, F., and Buckingham, M. (1999). From insect eye to vertebrate muscle: redeployment of a regulatory network. Genes Dev 13, 3171-8.

Represa, J., Frenz, D. A., and Van De Water, T. R. (2000). Genetic patterning of embryonic inner ear development. Acta Otolaryngol 120, 5-10.

Schafer, B. W., Czerny, T., Bernasconi, M., Genini, M., and Busslinger, M. (1994). Molecular cloning and characterization of a human PAX-7 cDNA expressed in normal and neoplastic myocytes. Nucleic Acids Res 22, 4574-82.

Schwarz, M., Alvarez-Bolado, G., Urbanek, P., Busslinger, M., and Gruss, P. (1997). Conserved biological function between Pax-2 and Pax-5 in midbrain and cerebellum development: evidence from targeted mutations. Proc Natl Acad Sci USA 94, 14518-23.

Seale, P., Sabourin, L. A., Girgis-Gabardo, A., Mansouri, A., Gruss, P., and Rudnicki, M. A. (2000). Pax7 is required for the specification of myogenic satellite cells [In Process Citation]. Cell 102, 777-86.

Soneoka, Y., Cannon, P. M., Ramsdale, E. E., Griffiths, J. C., Romano, G., Kingsman, S. M., and Kingsman, A. J. (1995). A transient three-plasmid expression system for the production of high titer retroviral vectors. Nucleic Acids Res 23, 628-33.

Sosa-Pineda, B., Chowdhury, K., Torres, M., Oliver, G., and Gruss, P. (1997). The Pax4 gene is essential for differentiation of insulin-producing beta cells in the mammalian pancreas. Nature 386, 399-402.

St-Onge, L., Sosa-Pineda, B., Chowdhury, K., Mansouri, A., and Gruss, P. (1997). Pax6 is required for differentiation of glucagon-producing alpha-cells in mouse pancreas. Nature 387, 406-9.

Strachan, T., and Read, A. P. (1994). PAX genes. Curr Opin Genet Dev 4, 427-38.

Tajbakhsh, S., Rocancourt, D., Cossu, G., and Buckingham, M. (1997). Redefining the genetic hierarchies controlling skeletal myogenesis: Pax-3 and Myf-5 act upstream of MyoD. Cell 89, 127-38.

Torban, E., Eccles, M. R., Favor, J., and Goodyer, P. R. (2000). PAX2 suppresses apoptosis in renal collecting duct cells. Am J Pathol 157, 833-42.

Tremblay, P., Dietrich, S., Mericskay, M., Schubert, F. R., Li, Z., and Paulin, D. (1998). A crucial role for Pax3 in the development of the hypaxial musculature and the long-range migration of muscle precursors. Dev Biol 203, 49-61.

Williams, B. A., and Ordahl, C. P. (1994). Pax-3 expression in segmental mesoderm marks early stages in myogenic cell specification. Development 120, 785-96.

Wilm, B., Dahl, E., Peters, H., Balling, R., and Imai, K. (1998). Targeted disruption of Pax1 defines its null phenotype and proves haploinsufficiency. Proc Natl Acad Sci USA 95, 8692-7.

1. A vector comprising an expression cassette comprising a sequence encoding a Pax protein, wherein the Pax protein is selected from the groups consisting of: Pax7; Pax3; an active variant of Pax 7; an active variant of Pax 3; an active fragment of Pax 7; and an active fragment of Pax 7, and wherein the Pax protein can induce myogenic differentiation of adult pluripotent stem cells.

2. A method of differentiating adult pluripotent stem cells to produce myoblasts comprising the step of transforming or infecting the stem cells with a vector comprising an expression cassette comprising a sequence encoding a Pax protein, wherein the Pax protein is selected from the groups consisting of: Pax7; Pax3; an active variant of Pax 7; an active variant of Pax 3; an active fragment of Pax 7; and an active fragment of Pax 7.

3. A method of treating a patient, comprising transplanting myoblasts produced according to the method of claim 2 into said patient.

4. The use according to claim 3, wherein said mammal is a human.
